(19) United States
(12) Patent Application Publication
(10) Pub. No.: US 2025/0135072 A1
(43) Pub. Date: May 1, 2025

(54) COMPOSITIONS COMPRISING A BIOABSORBABLE POLYMER AND METABOLIC INHIBITOR

(71) Applicant: Board of Trustees of Michigan State University, East Lansing, MI (US)

(72) Inventors: Chima V. MADUKA, Westminster, CO (US); Christopher CONTAG, East Lansing, MI (US)

(73) Assignee: Board of Trustees of Michigan State University, East Lansing, MI (US)

(21) Appl. No.: 18/833,687

(22) PCT Filed: Jan. 27, 2023
(86) PCT No.: PCT/US2023/011733

§ 371 Date: Jul. 26, 2024

Related U.S. Application Data
(60) Provisional application No. 63/303,757, filed on Jan. 27, 2022.

(51) Int. Cl. A61L 27/54; A61L 27/48; C08L 67/04

(52) U.S. Cl. CPC A61L 27/54; A61L 27/48; C08L 67/04; A61L 2300/206; A61L 2300/432

(57) ABSTRACT

Disclosed herein are compositions comprising a polymer and a metabolic inhibitor, as well as a method of using the composition to modulate an immune response. The composition may be produced in the form of a synthetic tissue for provision in a subject. The composition or synthetic tissue may further comprise additional therapeutic agents.

### CROSS-REFERENCE TO RELATED APPLICATIONS

This application claims the benefit under 35 U.S.C. § 119 (e) to U.S. Provisional Application No. 63/303,757 filed on 27 Jan. 2022. The entire contents of U.S. 63/303,757 are hereby incorporated by reference in their entirety.

### FIELD

The field of the invention relates to compositions comprising a polymer and a metabolic inhibitor. The composition may be used as a synthetic tissue that may modulate an immune response.

### BACKGROUND

The background description includes information that may be useful in understanding the compositions and methods described herein. It is not an admission that any of the information provided herein is prior art or relevant to the compositions and methods, or that any publication specifically or implicitly referenced is prior art.

Polylactide (PLA) is the most widely utilized biopolymer, with applications in nanotechnology, drug delivery, and adult reconstructive surgery for tissue regeneration. However, after surgical implantation, all synthetic biopolymers, including PLA, may elicit adverse immune responses in human and veterinary patients, which limits their use and often requires further interventions including removal of the biopolymer. In animal models excessive fibrosis results from long-term inflammation including those caused by PLA or other synthetic polymers, which significantly limits implant-tissue integration. PLA in vivo degrades by hydrolysis into D- or L-lactic acid monomers or oligomers, with semi-crystalline PLA degrading slower and tending to contain less D-lactic acid than amorphous PLA. Adverse responses to PLA and its breakdown components are exacerbated by mechanical loading and increasing implant size and occur after prolonged exposure to large amounts of PLA degradation products.

Adverse responses have been thought to be mediated by a reduction in pH in surrounding tissue due to PLA degradation. Establishing that a decrease in pH correlates with PLA degradation has informed previous strategies in regenerative medicine to neutralize acidic PLA degradation products both in vitro and in vivo using polyphosphazene, calcium carbonate, sodium bicarbonate and calcium hydroxyapatite salts, bioglass, and composites containing alloys or hydroxides of magnesium. Despite significant efforts using these approaches, each has led to a failure to control inflammatory responses. The lack of a clearly described mechanism of immune cell activation by PLA degradation had remained a major obstacle in the safe application of large PLA-based implants in load-bearing applications as reflected by the paucity of FDA approvals for large PLA implants and use in soft tissue surgery where neutralizing ceramics cannot be applied.

Metabolic reprogramming refers to significant changes in oxidative phosphorylation and glycolytic flux patterns and is a driver of fibrosis and bacterial lipopolysaccharide (LPS)-induced inflammation. The role that metabolic reprogramming and altered bioenergetics (ATP levels) plays in immune responses to PLA had yet to be established prior to the inventor's discovery.

### SUMMARY

A composition comprising a polymer and a metabolic inhibitor is disclosed herein. The polymer may be a bioabsorbable polymer (such as PLA, poly (lactide-co-glycolide) (PLGA), polyglycolide (PGA), and their copolymers) or a mixture of two or more polymers. The metabolic inhibitor may be a 6-phosphofructo-2-kinase/fructose-2,6-bisphosphatase 3 (PFKFB3) inhibitor, a glycolytic inhibitor, a biguanide, a γ-aminobutyrate aminotransferase (GABA-T) inhibitor, an inhibitor of the mitochondrial electronic transport chain (mETC inhibitor), or a combination of one or more of these or other metabolic inhibitors. The composition may be in the form of a synthetic tissue (e.g., a synthetic bone, synthetic cartilage, synthetic tendon, synthetic skin, synthetic blood, synthetic kidney, or synthetic liver). Alternatively, the composition may be in the form of a depot, such as a drug depot, in which metabolic inhibitor is released from the depot as the polymer breaks down. The composition may further include an additional therapeutic agent.

A method for modulating an immune response is also described herein. The method comprises providing to a subject in need thereof (e.g., a patient experiencing an undesired immune response), a composition comp rising a polymer and a metabolic inhibitor. The polymer may be a PLA, a PLGA, a PGA, or a combination thereof. The metabolic inhibitor may be a PFKFB3 inhibitor, a glycolytic inhibitor, a biguanide, a GABA-T inhibitor, an mETC inhibitor, or a combination of one or more of the metabolic inhibitors. The composition provided to the subject may be in the form of a synthetic tissue (e.g., a synthetic bone, synthetic cartilage, synthetic tendon, synthetic skin, blood, kidney, or liver). Alternatively, the subject may receive a depot, such as a drug depot, in which a metabolic inhibitor is released from the depot as the polymer breaks down. The method may further include an additional therapeutic agent. The subject may be a human or a non-human animal.

A synthetic tissue comprising a polymer and a metabolic inhibitor is also described herein. The synthetic tissue is designed to mimic an existing or removed body part in size, structure and/or function. The synthetic tissue may be in the form of a bone, cartilage, tendon, skin, blood, kidney, or liver. The polymer of the synthetic tissue may be a PLA, a PLGA, a PGA, or a combination thereof. The metabolic inhibitor may be a PFKFB3 inhibitor, a glycolytic inhibitor, a biguanide, a GABA-T inhibitor, an mETC inhibitor, or a combination of one or more of the metabolic inhibitors.

Depots, such as drug depots, comprising a polymer and a metabolic inhibitor are also described herein. The depots do not take on the size, structure, and/or function of a body part and are provided in a therapeutically suitable location of the body, including but not limited to the vascular system. The depot breaks down over time to release the metabolic inhibitor and, in some embodiments, an additional therapeutic agent. The polymer of the depot may be a PLA, a PLGA, a PGA, or a combination thereof. The metabolic inhibitor may be a PFKFB3 inhibitor, a glycolytic inhibitor, a biguanide, a GABA-T inhibitor, an mETC inhibitor, or a combinations of one or more of the metabolic inhibitors.

Various objects, features, aspects, and advantages will become more apparent from the following detailed description of preferred embodiments, along with the accompanying drawings.

### BRIEF DESCRIPTION OF THE DRAWINGS

FIG. 1 depicts ATP levels in mouse embryonic fibroblasts (MEFs) following exposure to crystalline or amorphous PLA over twelve days.

FIG. 2 depicts ATP levels in MEF cell lysates following culturing the cells in crystalline or amorphous PLA for seven or twelve days.

FIG. 3 depicts ATP levels in MEF cell lysates following seven or twelve days of culturing in the absence of PLA.

FIG. 4 depicts in-culture glucose levels in cell culture medium after incubation with crystalline or amorphous PLA over twelve days followed by addition of crystalline or amorphous PLA.

FIG. 5 depicts total ATP levels in primary bone marrow-derived macrophages (BMDMs) following prolonged exposure to crystalline or amorphous PLA.

FIG. 6 depicts total ADP levels in primary BMDMs following prolonged exposure to crystalline or amorphous PLA.

FIG. 7 depicts the ATP/ADP ratio and in-culture glucose levels in primary BMDMs following prolonged exposure to crystalline or amorphous PLA.

FIG. 8 depicts viability of primary BMDMs following prolonged exposure to crystalline or amorphous PLA.

FIG. 9 depicts MEF viability following prolonged exposure to crystalline or amorphous PLA.

FIG. 10 depicts the oxygen consumption rate (OCR) of primary BMDMs following exposure to amorphous PLA in the absence and presence of various metabolic inhibitors.

FIG. 11 depicts the extracellular acidification rate (ECAR) of primary BMDMs following exposure to amorphous PLA in the absence and presence of various metabolic inhibitors.

FIG. 12 depicts the proton efflux rate (PER) of primary BMDMs following exposure to amorphous PLA in the absence and presence of various metabolic inhibitors.

FIG. 13 depicts the OCR of primary BMDMs following exposure to crystalline PLA in the absence and presence of various metabolic inhibitors.

FIG. 14 depicts the ECAR of primary BMDMs following exposure to crystalline PLA in the absence and presence of various metabolic inhibitors.

FIG. 15 depicts the PER of primary BMDMs following exposure to crystalline PLA in the absence and presence of various metabolic inhibitors.

FIG. 16 depicts the ECAR of MEFs following exposure to amorphous or crystalline PLA.

FIG. 17 depicts the PER of MEFs following exposure to amorphous or crystalline PLA.

FIG. 18 depicts total ATP levels in MEFs following exposure to amorphous or crystalline PLA in the presence or absence of various metabolic inhibitors.

FIG. 19 depicts total ATP levels in primary BMDMs following prolonged exposure to monomeric L-lactic acid.

FIG. 20 depicts the ECAR, PER, and OCR in primary BMDMs following a seven-day culture in the presence of L-lactic acid.

FIG. 21 depicts the production of IL-6 (FIG. 21a), MCP-1 (FIG. 21b), TNF-α (FIG. 21c), IL-1B (FIG. 21d), IL-4 (FIG. 21e), and IL-10 (FIG. 21f) in primary BMDMs following a seven-day culture in the presence of amorphous or crystalline PLA and in the absence or presence of various metabolic inhibitors.

FIG. 22 depicts the increase in glucose uptake and inflammation due to PLA degradation in vivo. FIG. 22a shows radiolabeled glucose uptake. FIGS. 22b-e show the influx of macrophages to the surgical site of PLA implantation as shown by macrophage markers CD11b and F4/80, with decreased recruited proinflammatory macrophages (CD86) when PLA incorporates a metabolic inhibitor.

FIG. 23 depicts the activation of fibroblasts due to PLA degradation, in vivo. FIG. 23a shows a-SMA expression levels and FIG. 23b shows TGF-β expression levels.

FIG. 24 depicts the OCR of primary BMDMs following exposure to PLA of varied stereochemistries in the absence and presence of various metabolic inhibitors.

FIG. 25 depicts the ECAR of primary BMDMs following exposure to PLA of varied stereochemistries in the absence and presence of various metabolic inhibitors.

FIG. 26 depicts the PER of primary BMDMs following exposure to PLA of varied stereochemistries in the absence and presence of various metabolic inhibitors.

FIG. 27 depicts the total ATP levels in MEFs following exposure to PLA containing >99% L-lactide (PLLA), PLA containing >99% D-lactide (PDLA), and a 50/50 melt blend of PLLA and PDLA in the absence and presence of various metabolic inhibitors.

FIG. 28 depicts cytokine expression of BMDMs from untreated mice, mice treated with PLLA extract, mice treated with PDLA extract, or mice treated with a 50/50 melt-blend of PLLA and PDLA, including FIG. 28a: MCP-1; FIG. 28b: IL-1B; FIG. 28c: TNF-α; FIG. 28d: IL-6; FIG. 28e: IL-1B with inhibitors; FIG. 28f: TNF-α with inhibitors; FIG. 28g: IL-6 with inhibitors; FIG. 28h: IL-4; and FIG. 28i: IL-10 with inhibitors.

### DETAILED DESCRIPTION

### Definitions

The following definitions refer to the various terms used above and throughout the disclosure.

As used herein, all nouns in singular form are intended to convey the plural and all nouns in plural form are intended to convey the singular, except where context clearly indicates otherwise. As used herein, “and/or” includes any and all combinations of one or more of the associated listed items.

As used herein, “bioabsorbable polymer” refers to a polymer which can be broken down or degraded when exposed to and/or placed within a biological environment, such as on or within the body of an animal, including human, or placed under conditions that simulate or mimic a biological environment. The bioabsorbable polymer is not particularly limited, and may be a PLA, a PLGA, a PGA, or a combination thereof. Where the bioabsorbable polymer is a chiral molecule, such as with PLA, the bioabsorbable polymer encompasses racemic or stereocomplex mixtures of the polymer (e.g., a 50/50 mixture of both stereoisomers), or a mixture enriched for a specific stereoisomer (e.g., an 80/20 mixture of the D-and L-stereoisomers, respectively).

As used herein, “metabolic inhibitor” refers to a compound that inhibits, halts, or otherwise interferes with the ATP-producing pathways of a cell. The metabolic pathway encompasses numerous reaction schemes, such as glycolysis, glycogenolysis, fatty acid oxidation, amino acid oxidation, the Krebs cycle, the pentose phosphate pathway, and the electron transport system. A metabolic inhibitor is an agent that inhibits one or more steps of the one or more of the reaction schemes and reduces production of ATP. Metabolic inhibitors include, but are not limited to, PFKFB3 inhibitors that inhibit 6-phosphofructo-2-kinase/fructose-2,6-bisphosphatase 3, glycolytic inhibitors that inhibit glycolysis, biguanides that may enhance cellular glucose uptake to inhibit gluconeogenesis, and GABA-T inhibitors that inhibit γ-aminobutyrate aminotransferase. Specific PFKFB3 inhibitors may be 3-(3-pyridinyl)-1-(4-pyridinyl)-2-propen-1-one (3PO), (E)-1-(pyridin-4-yl)-3-(7-(trifluoromethyl)quinolin-2-yl)prop-2-en-1-one (ACT-PFK-158), (2S)-N-[4-[[3-cyano-1-[(3,5-dimethyl-4-isoxazolyl)methyl]-1H-indol-5-yl]oxy]phenyl]-2-pyrrolidine carboxamide (AZ76), (2S)-N-[4-[[3-cyano-1-(2-methylpropyl)-1H-indol-5-yl]oxy]phenyl]-2-pyrrolidine carboxamide (AZ 26), or 1-(4-pyridinyl)-3-(2-quinolinyl)-2-propen-1-one (PFK15). Specific glycolytic inhibitors may be 2-deoxyglucose (2DG), 3-bromopyruvate, 3-fluoro-1,2-phenylene bis(3-hydroxybenzoate) (WZB 117), 4-[[[[4-(1,1-dimethylethyl) phenyl]sulfonyl]amino]methyl]-N-3-pyridinylbenzamide (STF 31), phloretin, quercetin, dichloroacetate, oxamic acid, or NHI1. Specific biguanides may be metformin, buformin, or phenoformin. Specific GABA-T inhibitors may be aminooxyacetic acid, vigabatrin, gabaculine, phenelzine, phenylethylidinehydrazine (PEH), rosmarinic acid, valproic acid, ethanolamine-O-sulfate (EOS), or cycloserine. mETC inhibitors include rotenoids and macrolides. Specific rotenoids may be rotenone, rotenol, deguelin, dehydrodegulein, tephrosin, or sumatrol. Specific macrolides include oligomycin (e.g., oligomycin A, oligomycin B, oligomycin C, oligomycin D, oligomycin E, oligomycin F, rutamycin B, 44-homooligomycin A, or 44-homooligomycin B), azithromycin, clarithromycin, or erythromycin. Other particular mETC inhibitors include trifluoromethoxy carbonylcyanide phenylhydrazone (FCCP), and related drugs.

Alternatively, the metabolic inhibitor may be something other than a small molecule inhibitor. For example, the metabolic inhibitor may be nucleic acid that inhibits expression of an enzyme involved in the metabolic pathway (e.g., mRNA. RNAi, siRNA, miRNA, dsRNA). Additionally or alternatively, the metabolic inhibitor may be a gene that encodes the production of a protein (e.g., an antibody or an antigen binding fragment) that inhibits an enzyme involved in the metabolic pathway. The gene may be a synthetic, engineered, or natural gene (e.g., DNA). In some embodiments, the metabolic inhibitor is the antibody or antigen binding fragment itself.

As used herein, “effective amount” refers to the amount, dosage, and/or dosage regime of the metabolic inhibitor in the composition, synthetic tissue, or depot that is sufficient to induce a desired clinical and/or therapeutic outcome. The effective amount may also refer to the amount, dosage, and/or dosage regime of an additional therapeutic agent.

As used herein, “immune response” represents the action of one or more components of an immune system in reaction to one or more stimuli. The immune response may occur within a body of an animal (e.g., a human), outside the body of an animal (e.g., an ex vivo tissue), or in an in vitro environment that mimics the immune response. The immune response includes both the innate and the adaptive immune systems. Modulating an immune response includes both enhancing an immune response or inhibiting an immune response. Enhancing an immune response may include increasing expression and/or release of pro-inflammatory cytokines (e.g., IL-1, TNF-α, IL-6, and IFN-γ), increasing the inflammatory activity of immune cells, decreasing expression and/or release of anti-inflammatory cytokines (e.g., IL-4, IL-13, IL-10, and TGF-β), and/or decreasing the inflammatory activity of regulatory cells. Inhibiting an immune response may include decreasing expression and/or release of pro-inflammatory cytokines, decreasing the inflammatory activity of immune cells, increasing expression and/or release of anti-inflammatory cytokines, and/or increasing the activity of regulatory cells.

As used herein, “synthetic tissue” refers to a composition that mimics the structure, shape, and/or function of an endogenous organ, tissue, cell, blood cell, body part, or part thereof. The synthetic tissue composition comprises a polymer and a metabolic inhibitor. The synthetic tissue may mimic one or more cells, organs, tissues, body part, or part thereof, including, but not limited to, bone, cartilage, tendons, skin, blood, kidney, and liver. The synthetic tissue may replace an organ, tissue, or body part that has been removed in a subject. Alternatively, the synthetic tissue may replace or fill in a void created by the absence of a portion of an organ, tissue, and/or body part. In such instances, the synthetic tissue may be grafted onto a damaged or partially removed organ, tissue, and/or body part. Alternatively, the synthetic tissue may be inserted into an area of the body that is lacking a particular organ, tissue, and/or body part. The synthetic tissue may further comprise an additional therapeutic agent.

A “depot” is distinguished from a synthetic tissue in that the depot does not mimic or replace the structure and/or function of an organ. A depot, then, is comprised of a polymer and a metabolic agent, wherein the metabolic agent is released as the polymer degrades or through osmotic pressure.

As used herein, “subject,” “individual,” and “patient” interchangeably refer to a mammal, preferably a human or a non-human primate, but also domesticated mammals (e.g., canine or feline), laboratory mammals (e.g., mouse, rat, rabbit, hamster, guinea pig), and agricultural mammals (e.g., equine, bovine, porcine, ovine). In certain embodiments, the subject can be human (e.g., adult male, adult female, adolescent male, adolescent female, male child, female child) under the care of a physician or other health worker. In certain embodiments the subject may not be under the care of a physician or other health worker. The subject may have undergone surgery, received orthopedic treatment, received ophthalmic treatment, or suffering from injury or chronic disease. Alternatively, where the subject is a laboratory mammal, the composition, synthetic tissue, or depot may be provided to the laboratory mammal to achieve a scientific understanding rather than a clinical benefit.

### Compositions

The compositions described herein may comprise a polymer, such as a bioabsorbable polymer, or a combination of bioabsorbable polymers, and one or more metabolic inhibitors. The bioabsorbable polymer may be one or more of PLA, PLA-copolymer, PLGA, PGA, or a combination thereof. The polymer or combination of polymers may be further combined with or mixed with biologically acceptable metals and/or ceramics. The metabolic inhibitor may be a PFKFB3 inhibitor, a glycolytic inhibitor, a biguanide, a GABA-T inhibitor, an mETC inhibitor, or combinations thereof. The polymer may include stereoisomers of the polymer. In particular, the polymer may be PLA. The composition may be enriched for specific stereoisomers of PLA, including L-PLA or D-PLA. Instead, the composition may include a racemic or non-racemic mixture of PLA. Where the PLA is enriched for a specific stereoisomer, the enriched stereoisomer may be greater than about 55%, greater than about 60%, greater than about 65%, greater than about 70%, greater than about 80%, greater than about 85%, greater than about 90%, greater than about 95%, greater than about 96%, greater than about 97%, greater than about 98%, or greater than about 99% of the polymers in the composition.

The composition may comprise an amount of the metabolic inhibitor as low as about 1 μM and as high as about 1 M, or any amount in between, such as, about 10 μM, about 100 μM, about 0.25 mM, about 0.5 mM, about 1 mM, about 10 mM, about 15 mM, about 20 mM, about 25 mM, about 50 mM, and about 100 mM. The composition may comprise a range of metabolic inhibitor, such as between about 1 μM and about 1 M. Any range in between about 1 μM and about 1 M is also contemplated, including, but not limited to, between about 10 μM and about 100 mM, between about 100 μM and about 20 mM, or, in particular, between about 0.5 mM and about 15 mM.

The composition may comprise a PFKFB3 inhibitor as the metabolic inhibitor. The PFKFB3 inhibitor may be one or more of 3-(3-pyridinyl)-1-(4-pyridinyl)-2-propen-1-one (3PO), (E)-1-(pyridin-4-yl)-3-(7-(trifluoromethyl)quinolin-2-yl) prop-2-en-1-one (ACT-PFK-158), (2S)-N-[4-[[3-cyano-1-[(3,5-dimethyl-4-isoxazolyl)methyl]-1H-indol-5-yl]oxy]phenyl]-2-pyrrolidine carboxamide (AZ76), (2S)-N-[4-[[3-cyano-1-(2-methylpropyl)-1H-indol-5-yl]oxy]phenyl]-2-pyrrolidine carboxamide (AZ 26), or 1-(4-pyridinyl)-3-(2-quinolinyl)-2-propen-1-one (PFK15).

In a preferred embodiment, the metabolic inhibitor is 3PO and accounts for between about 0.02 and about 21 wt % of the total weight of the composition.

The composition may comprise a glycolytic inhibitor as the metabolic inhibitor. The glycolytic inhibitor may be one or more of 2-deoxyglucose (2DG), 3-bromopyruvate, 3-fluoro-1,2-phenylene bis(3-hydroxybenzoate) (WZB 117), 4-[[[[4-(1,1-dimethylethyl)phenyl]sulfonyl]amino]methyl]-N-3-pyridinylbenzamide (STF 31), phloretin, quercetin, dichloroacetate, oxamic acid, or NHI1. In a preferred embodiment, the metabolic inhibitor is 2DG and accounts for between about 0.01 and about 17 wt % of the total weight of the composition.

The composition may comprise a biguanide as the metabolic inhibitor. The biguanide may be one or more of metformin, buformin, or phenoformin. In a preferred embodiment, the metabolic inhibitor is metformin and accounts for between about 0.01 and about 11 wt % of the total weight of the composition.

The composition may comprise a GABA-T inhibitor as the metabolic inhibitor. The GABA-T inhibitor may be one or more of aminooxyacetic acid, vigabatrin, gabaculine, phenelzine, phenylethylidinehydrazine (PEH), rosmarinic acid, valproic acid, ethanolamine-O-sulfate (EOS), and cycloserine. In a preferred embodiment, the metabolic inhibitor is metformin and accounts for between about 0.01 and about 13 wt % of the total weight of the composition.

The composition may comprise a mETC inhibitor as the metabolic inhibitor. The mETC may be a rotenoid or a macrolide as the metabolic inhibitor. The rotenoid may be one or more of rotenone, rotenol, deguelin, dehydrodegulein, tephrosin, or sumatrol. The macrolide may be one or more of oligomycin, azithromycin, clarithromycin, or erythromycin. Alternatively, the mETC inhibitor may be a trifluoromethoxy carbonylcyanide phenylhydrazone (FCCP) or related drugs. In a preferred embodiment, the metabolic inhibitor is oligomycin and accounts for between about 0.0001 and about 13 wt % of the total weight of the composition.

Alternatively, the metabolic inhibitor can comprise a weight percentage (wt %) of the total weight of the composition, for example between about 0.01 and about 30 wt % of the total weight of the composition. The composition may comprise a weight percentage of any amount between the about 0.01 and about 30 wt %, including, but not limited to, about 0.05 wt %, about 0.1 wt %, about 0.2 wt %, about 0.5 wt %, about 1 wt %, about 2 wt %, about 3 wt %, about 4 wt %, about 5 wt %, about 6 wt %, about 7 wt %, about 8 wt %, about 9 wt %, about 10 wt %, about 11 wt %, about 12 wt %, about 13 wt %, about 14 wt %, about 15 wt %, about 16 wt %, about 17 wt %, about 18 wt %, about 19 wt %, about 20 wt %, about 21 wt %, about 22 wt %, about 23 wt %, about 24 wt %, about 25 wt %, about 26 wt %, about 27 wt %, about 28 wt %, or about 29 wt %. Any range between the about 0.01 wt % and about 30 wt % is also contemplated, including, but not limited to between about 0.02 to about 21 wt %, between about 0.01 and about 17 wt %, between about 0.01 and about 11 wt %, and between about 0.01 and about 13 wt % of the total weight of the composition.

Still further, the metabolic inhibitor may be uniformly spread throughout the composition providing a constant release of the metabolic inhibitor as the polymer degrades or breaks down. Alternatively, the metabolic inhibitor may be non-uniformly spread throughout the composition, providing a variable release of the metabolic inhibitor as the polymer degrades. For example, the metabolic inhibitor may be present at a relatively high weight percentage near the surface of the composition and at a relatively low weight percentage within the interior of the composition. In another embodiment, the metabolic inhibitor may be present at a relatively low weight percentage near the surface of the composition and at a relatively high weight percentage within the interior of the composition.

The weight percentage of the metabolic inhibitor and/or the specific polymer used in the composition may be standard for all patients or tailored (or otherwise custom set) for a particular patient in light of one or more of the condition of the particular patient, whether the patient is immunocompromised or generates a robust immune response, the particular genetic profile of the patient (including but not limited to the specific alleles and/or genetic predisposition of the patient), and/or the particular disease, disorder, or condition of the subject requiring treatment. The particular identity and weight percentage of the metabolic inhibitor and/or particular polymer to be used can be determined by the artisan.

In some embodiments, one or more metabolic inhibitors may be incorporated within the polymer, such as within the matrix of the polymer. Alternatively, one or more metabolic inhibitors may be coated on the exterior of the polymer. In some instances, one or more metabolic inhibitors may be both incorporated within the polymer and coated onto the exterior of the polymer.

The composition may vary according to how the composition is to be used. For example, the composition may be incorporated into a synthetic tissue, such as a synthetic bone, cartilage, tendon, skin, blood, kidney, and liver. Furthermore, the composition may contain an additional therapeutic agent. The additional therapeutic agent is not particularly limited and may be chemotherapies for cancer, antibiotics, small molecules, antibodies, antigens, calcium phosphate, hydroxyapatite, or bioglass.

### Methods of Use

The compositions described herein can be used in a variety of ways. In one embodiment is a method for modulating an immune response by providing the composition comprising a polymer, such as a bioabsorbable polymer or a combination of bioabsorbable polymers, and a metabolic inhibitor discussed above to a subject in need of such treatment. The bioabsorbable polymer may be one or more of PLA, PLA-copolymer, PLGA, PGA, PLA, or combinations thereof. The polymer or combination of polymers may be further combined with or mixed with biologically acceptable metals and/or ceramics. The metabolic inhibitor may be a PFKFB3 inhibitor, a glycolytic inhibitor, a biguanide, a GABA-T inhibitor, an mETC inhibitor, or a combination of one or more of the metabolic inhibitors. The metabolic inhibitor may be incorporated within the matrix of the polymer and/or coated onto the surface of the polymer. In a particular embodiment, the polymer may be PLA, including specific stereoisomers of PLA such as L-PLA, D-PLA, or a racemic or non-racemic mixture of PLA. Alternatively, the composition may be enriched for one particular stereoisomer of the polymer as described above. For example, the composition used in the method may comprise predominantly D-PLA or L-PLA.

In some embodiments, the composition is provided (e.g., inserted into the body of the subject) as a synthetic tissue. Alternatively, the composition is provided (e.g., inserted into the body of the subject) as a depot. Where the composition is in the form of a synthetic tissue, the synthetic tissue may supplement or replace bone, cartilage, tendon, skin, blood, kidney, and/or liver.

The composition provided to the subject may comprise between about 0.01 wt % and about 30 wt %, or any amount or range between these values. In particular, the metabolic inhibitor may account for between about 0.02 and about 21 wt %, between about 0.01 and about 17 wt %, between about 0.01 and about 11 wt %, and between about 0.01 and about 13 wt % of the total weight of the composition.

In some embodiments, the subject may have undergone surgery, received orthopedic treatment, received ophthalmologic treatment, or may be suffering from a chronic disease. The subject may be a human or a non-human animal.

### Synthetic Tissues and Depots

The compositions described herein can be prepared in the form of a synthetic tissue, wherein the synthetic tissue comprises a polymer (such as a bioabsorbable polymer or a combination of bioabsorbable polymers) and a metabolic inhibitor. The bioabsorbable polymer may be one or more of PLA, PLA-copolymer, PLGA, PGA, PLA, or combinations thereof. Alternatively, the polymer may further comprise the synthetic tissue. The metabolic inhibitor may be a PFKFB3 inhibitor, a glycolytic inhibitor, a biguanide, a GABA-T inhibitor, an mETC inhibitor, or a combination of one or more of the metabolic inhibitors. The synthetic tissue may be in the form of bone, cartilage, tendon, skin, blood, kidney, and/or liver. The synthetic tissue may completely replace an organ, tissue, or body part of a subject or may replace part of an organ, tissue, or body part. For example, the synthetic tissue may a synthetic bone in the form of a femur and provided to a subject whose femur, or portion thereof, has been removed. In an alternative example, the synthetic tissue may be in the form of a bone and grafted onto a subject's bone to replace a portion of the subject's bone, such as a diseased, damaged, and/or injured portion of bone that has been removed.

In particular, the polymer of the synthetic tissue may be PLA, including stereoisomers of PLA such as L-PLA, D-PLA, and racemic mixtures of PLA. Where the PLA is enriched for a specific stereoisomer, enriched stereoisomer may be greater than about 55%, greater than about 60%, greater than about 65%, greater than about 70%, greater than about 80%, greater than about 85%, greater than about 90%, greater than about 95%, greater than about 96%, greater than about 97%, greater than about 98%, or greater than about 99% of the polymers in the composition.

The synthetic tissue may comprise an amount of the metabolic inhibitor as low as about 1 μM and as high as about 1 M, or any amount in between, such as, about 10 μM, about 100 μM, about 0.25 mM, about 0.5 mM, about 1 mM, about 10 mM, about 15 mM, about 20 mM, about 25 mM, about 50 mM, and about 100 mM. The synthetic tissue may comprise a range of metabolic inhibitor, such as between about 1 μM and about 1 M. Any range in between about 1 μM and about 1 M is also contemplated, including, but not limited to, between about 10 μM and about 100 mM, between about 100 μM and about 20 mM, or, in particular, between about 0.5 mM and about 15 mM.

Alternatively, the metabolic inhibitor can comprise a weight percentage (wt %) of the total weight of the synthetic tissue, for example between about 0.01 and about 30 wt % of the total weight of the synthetic tissue. The composition may comprise a weight percentage of any amount between the about 0.01 and about 30 wt %, including, but not limited to, about 0.05 wt %, about 0.1 wt %, about 0.2 wt %, about 0.5 wt %, about 1 wt %, about 2 wt %, about 3 wt %, about 4 wt %, about 5 wt %, about 6 wt %, about 7 wt %, about 8 wt %, about 9 wt %, about 10 wt %, about 11wt %, about 12 wt %, about 13 wt %, about 14 wt %, about 15 wt %, about 16 wt %, about 17 wt %, about 18 wt %, about 19 wt %, about 20 wt %, about 21 wt %, about 22 wt %, about 23 wt %, about 24 wt %, about 25 wt %, about 26 wt %, about 27 wt %, about 28 wt %, or about 29 wt %. Any range between the about 0.01 wt % and about 30 wt % is also contemplated, including, but not limited to between about 0.02 to about 21 wt %, between about 0.01 and about 17 wt %, between about 0.01and about 11 wt %, and between about 0.01 and about 13 wt % of the total weight of the composition.

The synthetic tissue may comprise a PFKFB3 inhibitor as the metabolic inhibitor. The PFKFB3 inhibitor may be one or more of 3-(3-pyridinyl)-1-(4-pyridinyl)-2-propen-1-one (3PO), (E)-1-(pyridin-4-yl)-3-(7-(trifluoromethyl)quinolin-2-yl)prop-2-en-1-one (ACT-PFK-158), (2S)-N-[4-[[3-cyano-1-[(3,5-dimethyl-4-isoxazolyl)methyl]-1H-indol-5-yl]oxy]phenyl]-2-pyrrolidine carboxamide (AZ76), (2S)-N-[4-[[3-cyano-1-(2-methylpropyl)-1H-indol-5-yl]oxy]phenyl]-2-pyrrolidine carboxamide (AZ 26), or 1-(4-pyridinyl)-3-(2-quinolinyl)-2-propen-1-one (PFK15). In a preferred embodiment, the metabolic inhibitor is 3PO and accounts for between about 0.02 and about 21 wt % of the total weight of the synthetic tissue.

The synthetic tissue may comprise a glycolytic inhibitor as the metabolic inhibitor. The glycolytic inhibitor may be one or more of 2-deoxyglucose (2DG), 3-bromopyruvate, 3-fluoro-1,2-phenylene bis(3-hydroxybenzoate) (WZB 117), 4-[[[[4-(1,1-dimethylethyl) phenyl]sulfonyl]amino]methyl]-N-3-pyridinylbenzamide (STF 31), phloretin, quercetin, dichloroacetate, oxamic acid, or NHI1. In a preferred embodiment, the metabolic inhibitor is 2DG and accounts for between about 0.01 and about 17 wt % of the total weight of the synthetic tissue.

The synthetic tissue may comprise a biguanide as the metabolic inhibitor. The biguanide may be one or more of metformin, buformin, or phenoformin. In a preferred embodiment, the metabolic inhibitor is metformin and accounts for between about 0.01 and about 11 wt % of the total weight of the synthetic tissue.

The synthetic tissue may comprise a GABA-T inhibitor as the metabolic inhibitor. The GABA-T inhibitor may be one or more of aminooxyacetic acid, vigabatrin, gabaculine, phenelzine, phenylethylidinehydrazine (PEH), rosmarinic acid, valproic acid, ethanolamine-O-sulfate (EOS), and cycloserine. In a preferred embodiment, the metabolic inhibitor is metformin and accounts for between about 0.01 and about 13 wt % of the total weight of the synthetic tissue.

The synthetic tissue may comprise an mETC inhibitor as the metabolic inhibitor. The mETC inhibitor may be a rotenoid or a macrolide. The rotenoid may be one or more of rotenone, rotenol, deguelin, dehydrodegulein, tephrosin, or sumatrol. The macrolide may be one or more of oligomycin, azithromycin, clarithromycin, or erythromycin. Alternatively, the mETC inhibitor may be FCCP. In a preferred embodiment, the metabolic inhibitor is oligomycin and accounts for between about 0.0001 and about 13 wt % of the total weight of the synthetic tissue.

The metabolic inhibitor may be incorporated within the matrix of the polymer of the synthetic tissue. Alternatively, the metabolic inhibitor may be coated onto the surface of the polymer of the synthetic tissue. In another embodiment, the metabolic inhibitor may be both incorporated into the bioabsorbable matrix and coated onto the surface of the polymer of the synthetic tissue. In such instances, the metabolic inhibitor incorporated within the matrix of the polymer may be the same or different than the metabolic inhibitor coated onto the surface of the polymer. The particular species of metabolic inhibitor incorporated into the polymer matrix or coated onto the surface of the polymer can be determined to accommodate the particular requirements of a treatment protocol. An additional therapeutic agent may also be delivered if present in the synthetic tissue.

The synthetic tissue may be produced through three-dimensional printing technology. Following incorporation of the metabolic inhibitor in the polymer by melt-blending, filaments may be extruded. Computer-aided design, which may be patient-derived, utilize extruded filaments in a multi-dimensional printer to make synthetic tissues. Multiple dimensional printers may be 3D or 4D printers. Alternatively, printed polymeric implants may have their surfaces coated by methods not limited to physical or chemical binding.

Alternatively, the compositions described herein can be prepared in the form of a depot, which does not possess the shape, structure, or function of an organ, tissue, or body part of the subject. The size of the depot is not particularly limited and may range in diameter from a nanometer scale to a centimeter scale In this manner, the depot delivers one or more metabolic inhibitor to the subject as the polymer decomposes or the inhibitor is pushed out via osmotic pressure. An additional therapeutic agent may also be delivered if present in the depot. The depot can be inserted into the body cavity of a subject, such as under the skin or within a body cavity. The particular polymers and metabolic inhibitors for the depot are similar to those for the synthetic tissue described above.

### EXAMPLES

The following example is provided to further illustrate the fusion peptide disclosed herein but should not be construed as in any way limiting its scope.

### Example 1: PLA Degradation and Metabolic Remodeling

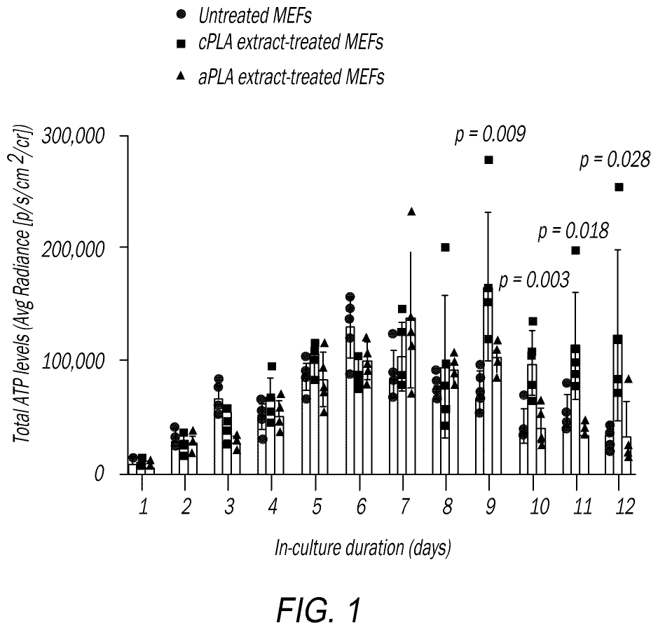

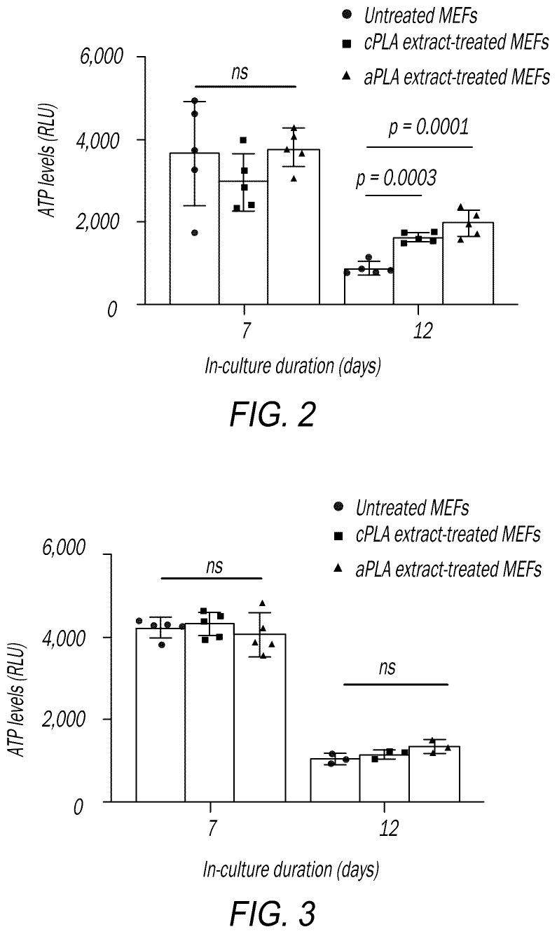

This example demonstrates the molecular mechanism underlying metabolic reprogramming in inflammation and fibrosis following degradation of the bioabsorbable polymer PLA. Breakdown products of PLA (hereafter “extracts”) were generated in serum-containing media and used after twelve days of incubation in a shaker at 37° C. The in vitro degradation mimics PLA degradation in vivo. Immune cells were maintained in the PLA extracts for twelve days. BMDMs derived from C57BL/6J mice (Jackson Laboratories) of 3-4 months were used. Mouse embryonic fibroblasts (NIH 3T3 cells, hereafter “MEFs”) were stably transfected with a Sleeping Beauty transposon plasmid (pLuBIG) encoding blasticidin resistance linked to eGFP, and luciferase. Bioluminescence of the fibroblasts served as in indicator of ATP levels. Cell viability was measured using the crystal violet staining assay at room temperature. 50 μL media containing cells is incubated with 150 μL of 99.9% methanol for 15 minutes. 100 μL of 0.5% crystal violet (25% methanol) is added for 20 minutes. Each well is emptied and washed twice with 200 μL phosphate buffered saline for 2 minutes. Absorbance (optical density) was acquired at 570 nm using the SpectraMax M3 Spectrophotometer (Molecular Devices) and SoftMax Pro software (ver. 7.0.2).

Whereas short-term exposure to amorphous or crystalline PLA (e.g., 0-5 days) induced no incremental changes in ATP levels, prolonged exposure (e.g., 6-12 days) of fibroblasts to either amorphous or crystalline PLA increased ATP levels in live cells (FIG. 1). Upon high resolution z-stack imaging, there were apparent changes in nucleoli number after prolonged exposure to either amorphous or crystalline PLA extract, which could represent a stress response. To exclude the possibility that changing luciferase expression (by transcription or translation) was responsible for observed bioenergetic changes, wild-type cells were lysed after exposure to PLA extract. Controlled amounts of luciferase and D-luciferin were added to the standard ATP assay. By day 12, there was a 1.9-and 2.3-fold increase in ATP levels among cells exposed to crystalline and amorphous PLA extract, respectively (FIG. 2). To exclude the possibility that PLA extracts affect the biochemical reaction underlying bioenergetic measurements, lysed fibroblasts were exposed to D-luciferin, luciferase and control or PLA extract. No difference in ATP levels was observed, confirming that treatment with PLA extract did not affect this biochemical reaction (FIG. 3).

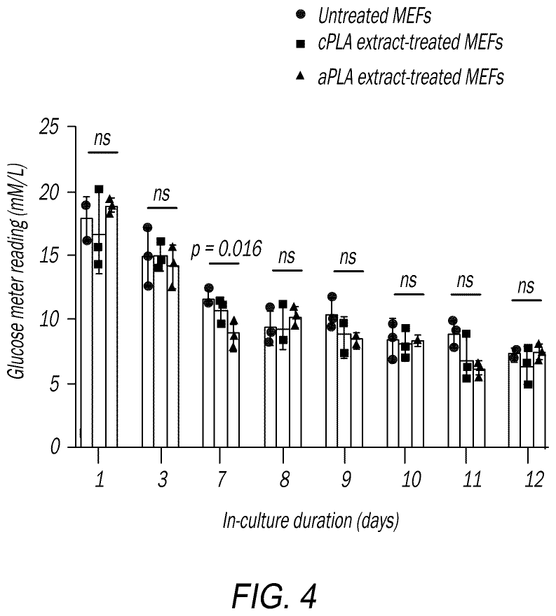

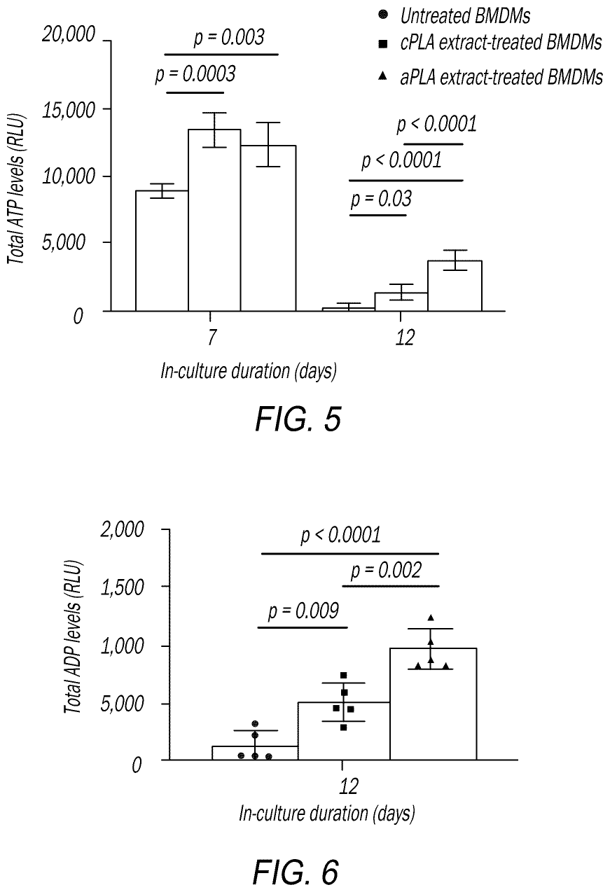

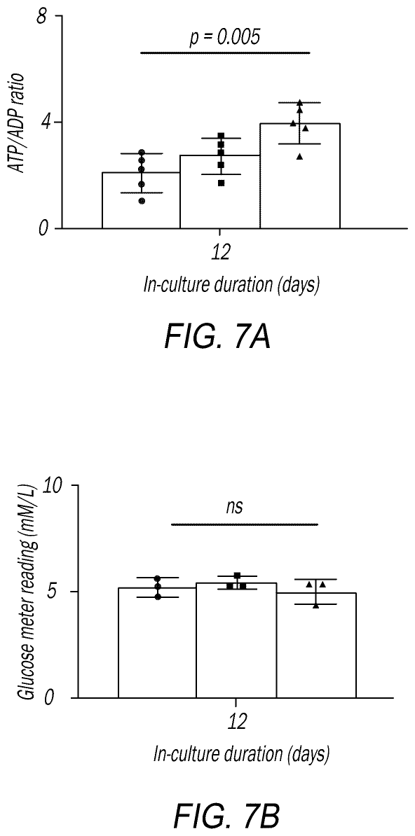

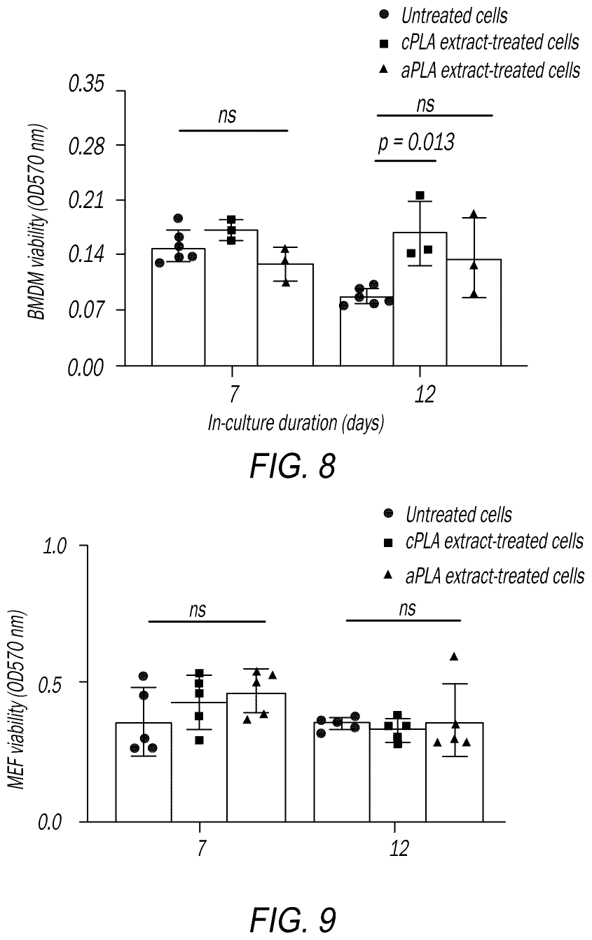

To determine whether glucose levels changed between groups on the same day because of the extended exposure times in our model, glucose meter readings were optimized in mammalian cell culture medium. Glucose levels were similar between the groups on each day (FIG. 4). On day 7, when untreated groups had higher glucose levels (FIG. 4), corresponding bioenergetic measurement revealed that PLA extract-treated fibroblasts had higher ATP levels, excluding changing glucose levels as a confounding factor in the bioenergetic model. Because MEFs are normal immortalized fibroblasts, changing cell number from in vitro proliferation could account for bioenergetic changes. To exclude this, a crystal violet assay for cell number measurement in fibroblasts was optimized. Next, primary BMDMs were isolated. BMDMs, unlike NIH 3T3 cells, do not proliferate. The BMDMs behaved like the fibroblasts and showed marked increases in ATP and ADP levels (FIGS. 5 and 6) or ATP/ADP ratios which were not due to changing glucose levels (FIG. 7). After optimizing the crystal violet assay for macrophages, there were no changes in cell numbers (FIG. 8). Furthermore, fibroblast numbers were similar for cultures that were untreated or exposed to PLA extracts (FIG. 9).

To determine the metabolic pathways responsible for the observed bioenergetic changes, Seahorse assays were used to measure oxygen consumption rate (OCR), extracellular acidification rate (ECAR) and lactate-linked proton efflux rate (PER) in a customized medium (pH 7.4). Basal measurements of OCR, ECAR, and PER were obtained in real time using the Seahorse XFe-96 Extracellular Flux Analyzer (Agilent Technologies). Prior to running the assay, cell culture medium was washed with and replaced by the Seahorse XF DMEM medium (pH 7.4) supplemented with 25 mM D-glucose and 4 mM glutamine. The Seahorse plates were equilibrated in a non-CO₂ incubator for an hour prior to the assay. The Seahorse ATP rate and cell energy phenotype assays were run according to manufacturer's instruction and all reagents for the Seahorse assays were sourced from Agilent Technologies. Wave software (Version 2.6.1) was used to export Seahorse data directly as means±standard deviation (SD).

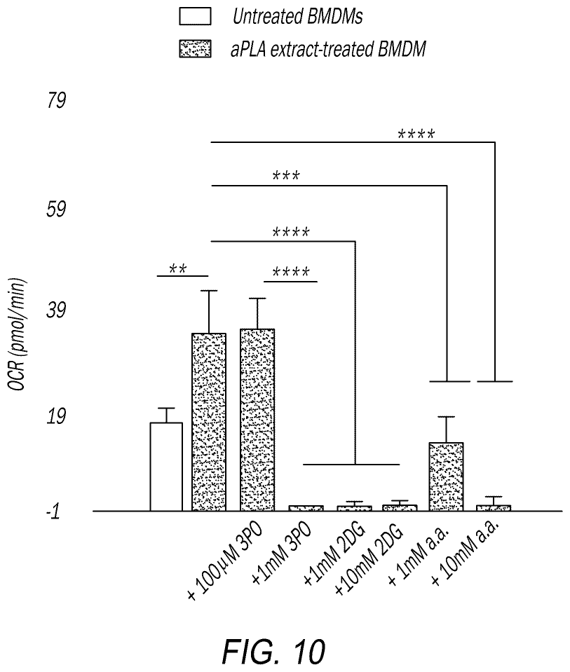

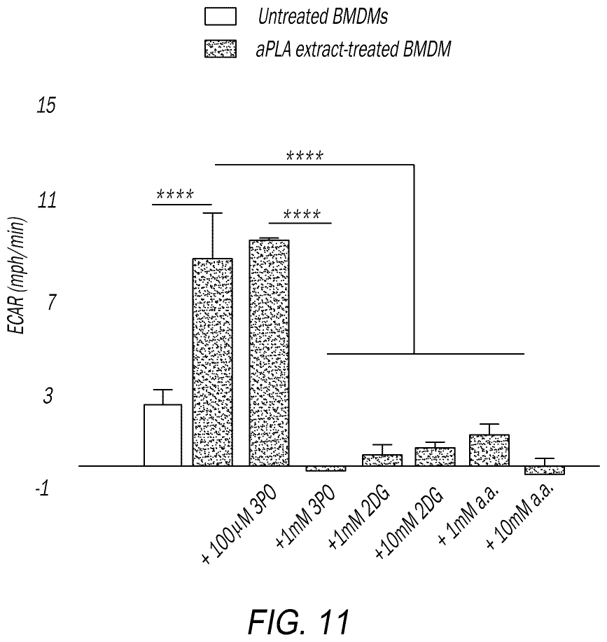

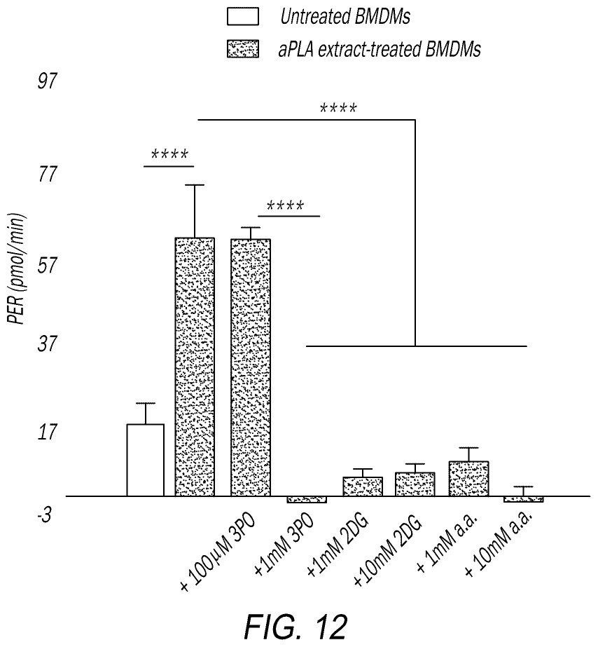

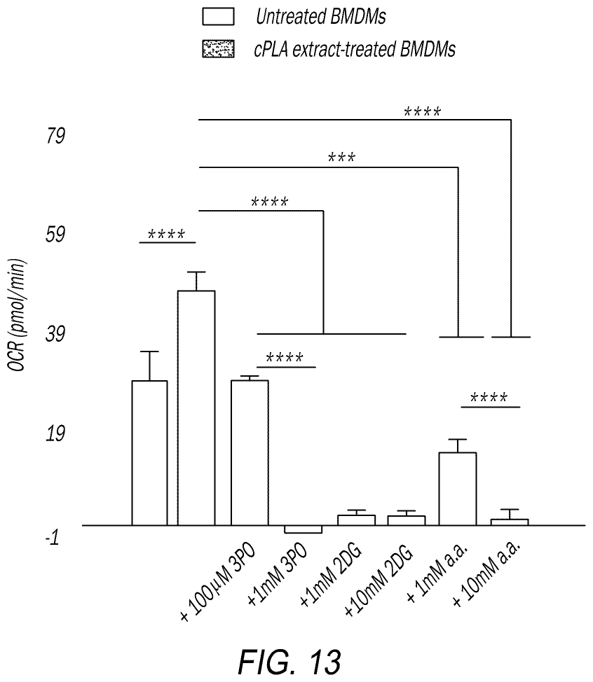

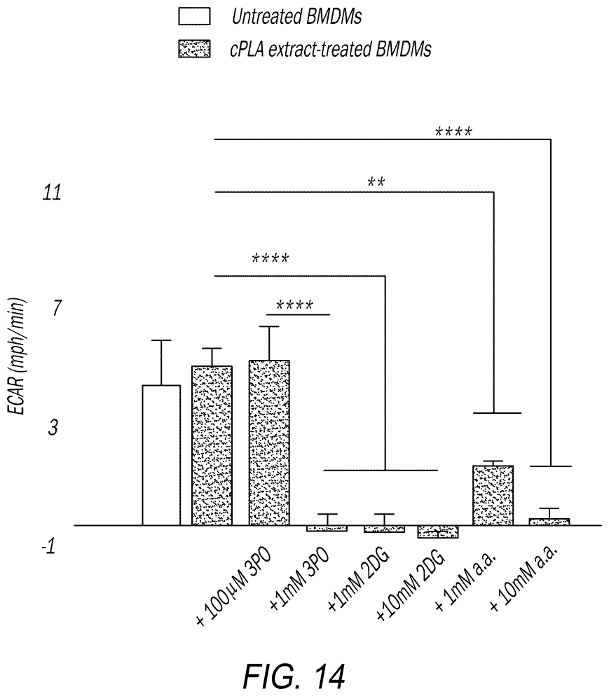

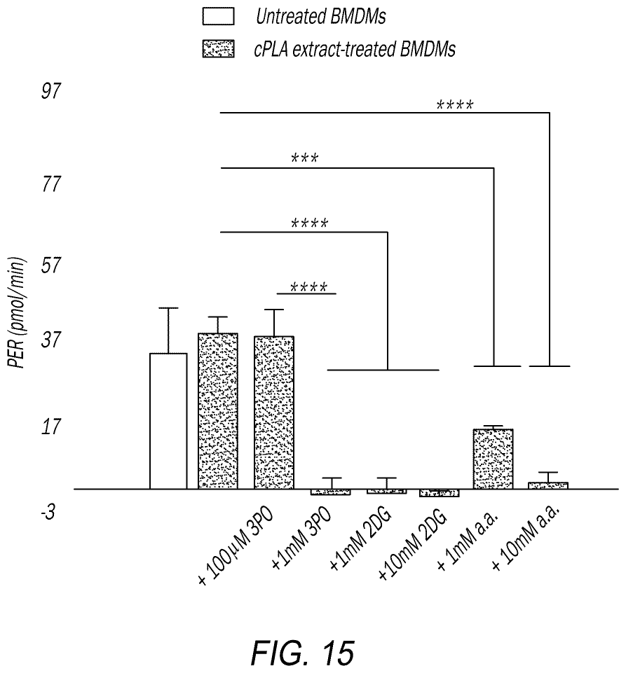

Seahorse assays measures ECAR as an index of glycolytic flux, OCR as an index of oxidative phosphorylation and PER as an index of monocarboxylate transporter function in live cells; and are used to assess for metabolic reprogramming. Primary BMDMs exposed to amorphous PLA extract were metabolically altered, showing a 2-fold increase in oxidative phosphorylation (OCR; FIG. 10), 3.5-fold increase in glycolytic flux (ECAR; FIG. 11) and 3.5-fold increase in monocarboxylate transporter activity (PER; FIG. 12) in comparison to untreated BMDMs. Similar amounts (100 μl) of crystalline PLA extract resulted in a 1.6-fold increase in OCR (FIG. 13) but no change in ECAR (FIG. 14) or PER (FIG. 15). However, higher amounts (150 μl) of crystalline PLA extract resulted in 3.2-, 3.8-, and 3.8-fold increases in OCR, ECAR and PER, respectively compared to controls, suggesting that greater volume of PLA extract is required for reprogramming using crystalline than amorphous PLA.

Dose-dependently, 3PO, 2DG and aminooxyacetic acid inhibited metabolic reprogramming following exposure to amorphous PLA or crystalline PLA extract but not in untreated BMDMs. This demonstrates cellular uptake of 3PO, 2DG and aminooxyacetic acid, yet with selective pharmacologic effects. Notably and under the same experimental conditions, cell viability was not reduced in untreated BMDMs after exposure to glycolytic inhibitors, demonstrating the absence of cytotoxicity. However, when BMDMs were treated with amorphous or crystalline PLA extract, where metabolism was abnormally remodeled, 3PO, 2DG and aminooxyacetic acid selectively reduced cell viability. Therefore, pharmacologically targeting altered metabolism in primary BMDMs following exposure to PLA extract is highly specific with limited toxicity to immune cells that have normal metabolic profiles.

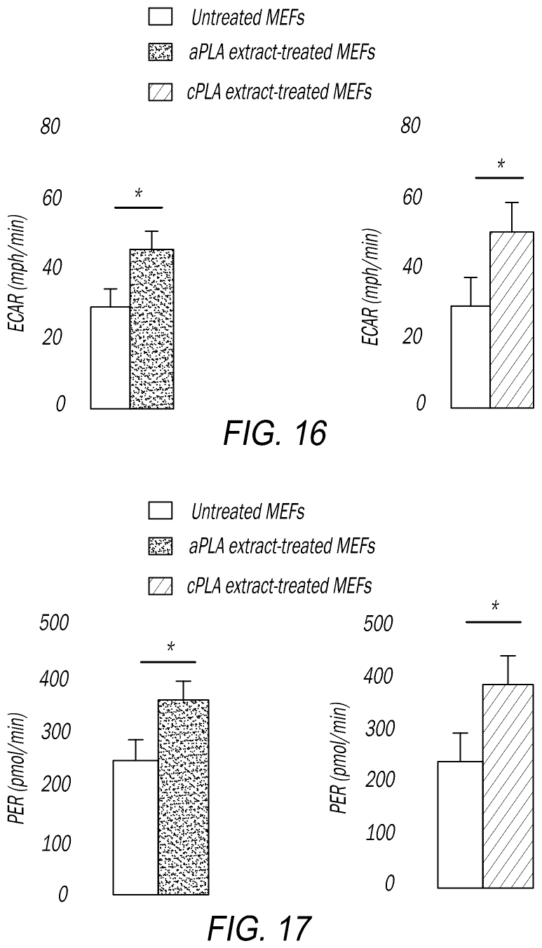

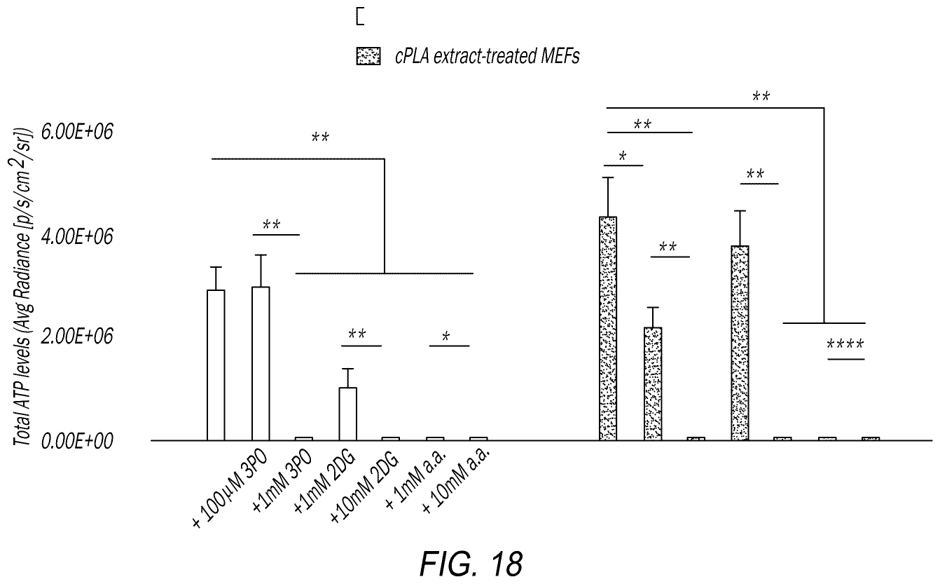

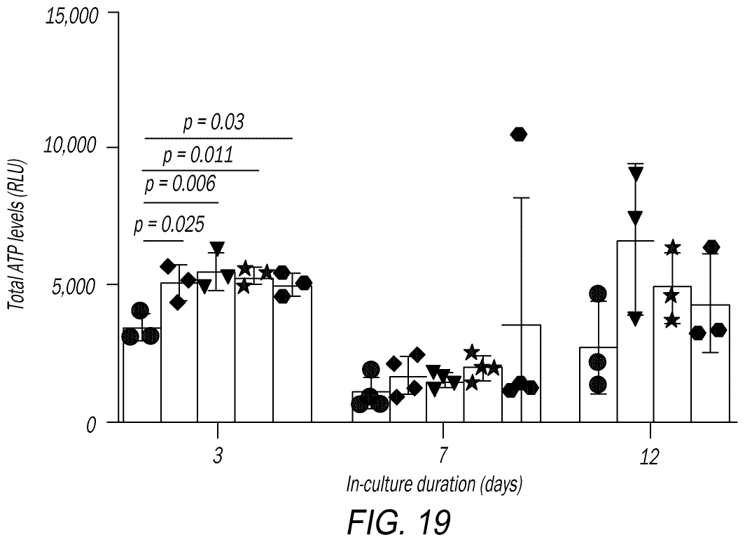

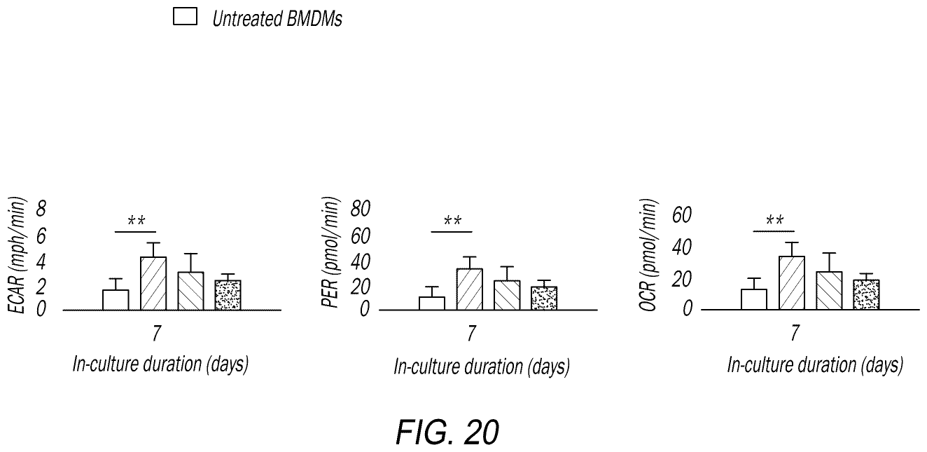

After prolonged exposure of fibroblasts to amorphous and crystalline PLA extracts, glycolytic flux (ECAR; FIG. 16) is increased by 1.6- and 1.7-fold, respectively. Furthermore, monocarboxylate transporter function is increased in amorphous or crystalline PLA extract-treated fibroblasts by 1.6- and 1.5-fold, respectively (FIG. 17). However, oxidative phosphorylation remains similar between untreated fibroblasts and cells exposed to amorphous or crystalline PLA extracts. Remarkably, increased bioenergetic (ATP) levels in amorphous or crystalline PLA extract-treated fibroblasts are inhibited by 3PO, 2DG and aminooxyacetic acid in a spatiotemporal and dose-dependent manner (FIG. 18). There was no reduction in mass of PLA after 12 day extraction, but there were detectable changes in molecular weight. There was a 2.7- and 2.8-fold increase in D-lactic acid in amorphous and crystalline PLA extracts, respectively. BMDMs were exposed to various doses of L-lactic acid, ranging from 2.5- to 15-fold higher levels in comparison to untreated cells. Bioenergetic levels were altered in the short-term (day 3) for all doses of L-lactic acid treatment, resulting in a 1.5 to 1.6-fold increase in ATP levels (FIG. 19). After prolonged (day 7) exposure to L-lactic acid and even when bioenergetic alterations were not apparent, glycolytic flux (ECAR), monocarboxylate transporter function (PER) and oxidative phosphorylation (OCR) were increased by 2.8-, 2.8- and 2.3-fold, mechanistically reproducing observations made with amorphous and crystalline PLA extracts in our bioenergetic model (FIG. 20). These changes were not dependent on alterations in cell number. Highly acidic groups (5-15 mM L-lactic acid) did not result in reduction in viability of primary macrophages either at day 7 or 12, relative to controls.

Cytokine and chemokine levels were measured using a MILLIPLEX MAP mouse magnetic bead multiplex kit (MilliporeSigma) to assess for IL-6, MCP-1, TNF-a, IL-1b, IL-4, IL-10, IFN-1 and 1L-13 protein expression in supernatants. Data was acquired using Luminex 200 (Luminex Corporation) by the xPONENT software (Version 3.1, Luminex Corporation). Using the glycolytic inhibitor, 3PO, expectedly decreased cytokine values to <3.2 pg/mL in some experiments. For statistical analyses, those values were expressed as 3.1 pg/mL. Values exceeding the dynamic range of the assay, in accordance with manufacturer's instruction, were excluded. Additionally, IL-6 ELISA kits (RayBiotech) for supernatants were used according to manufacturer's instructions.

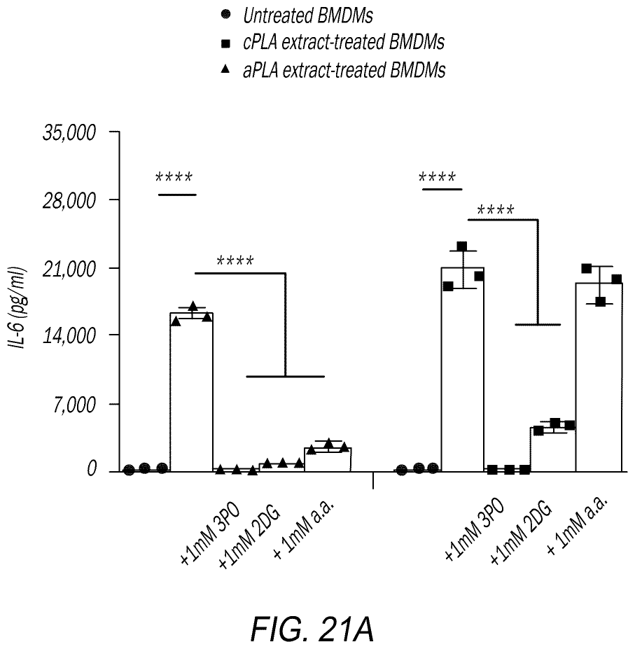

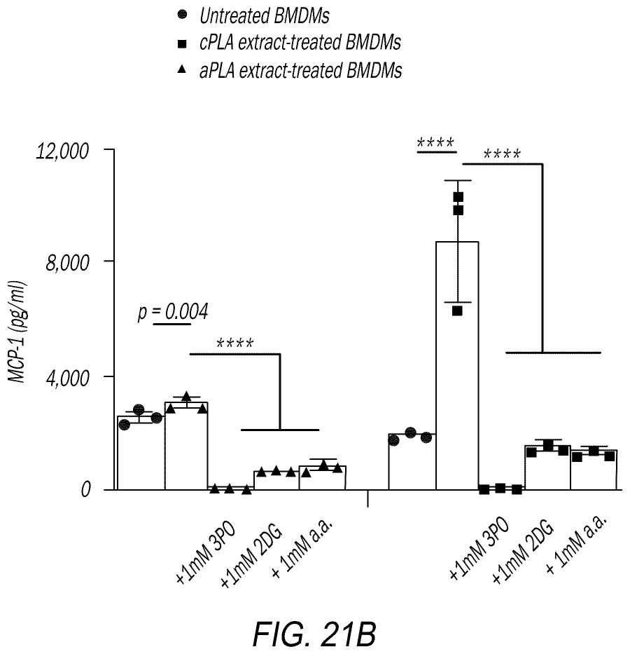

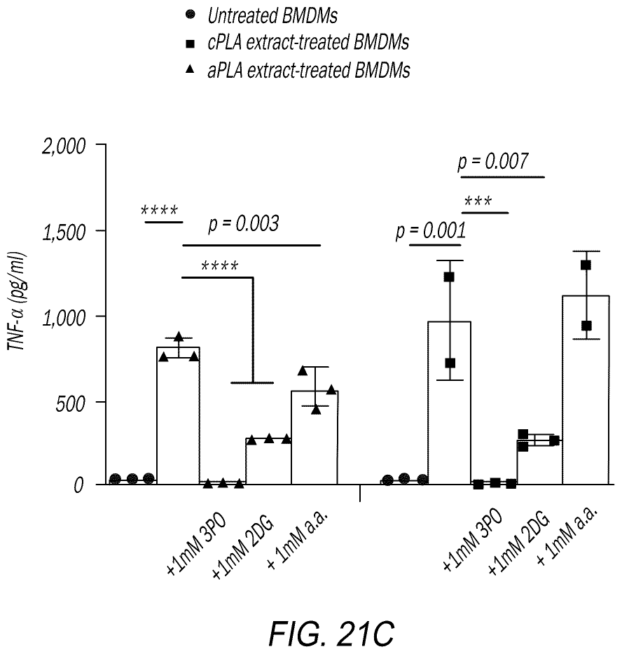

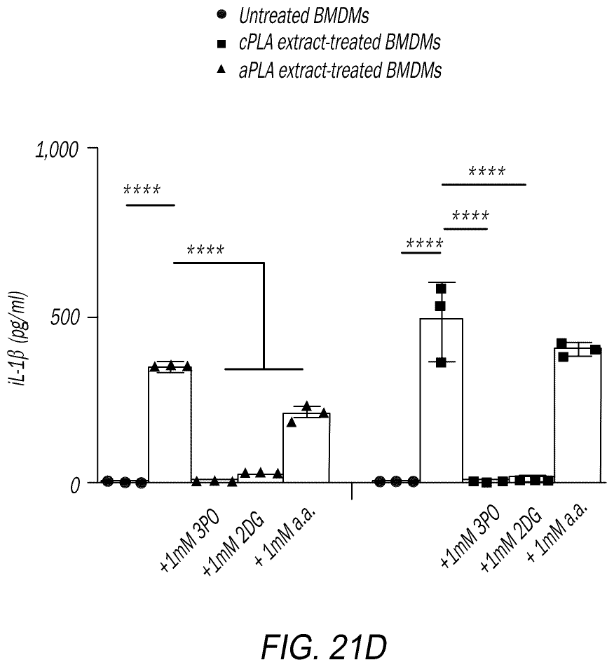

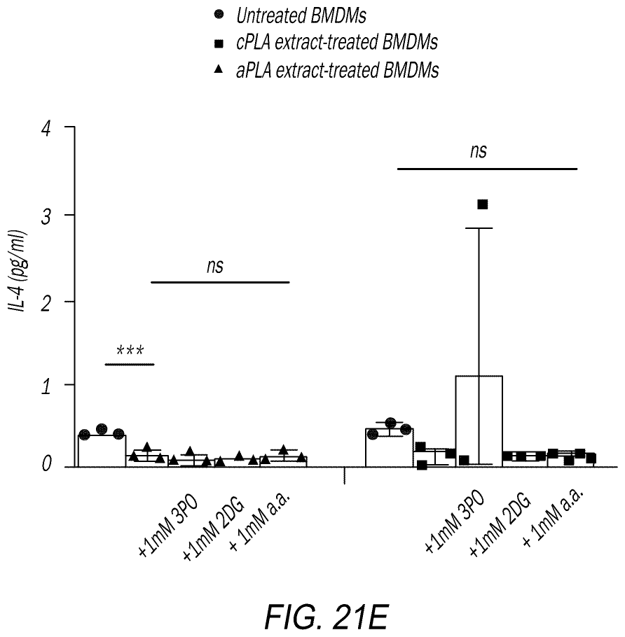

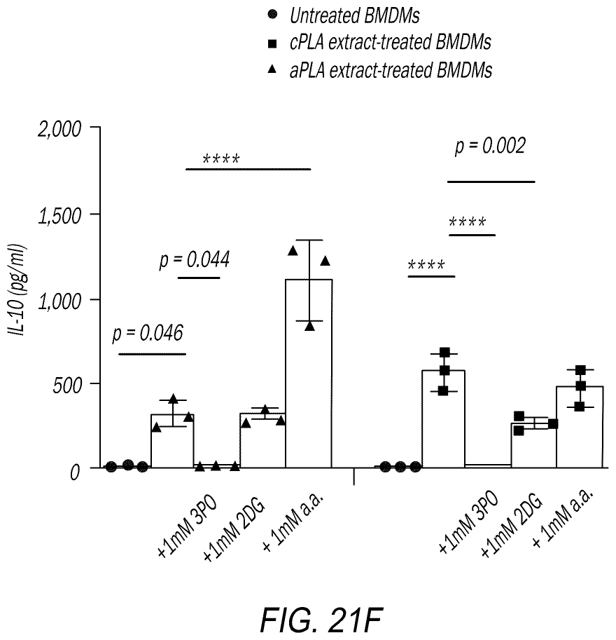

Prolonged exposure of primary macrophages to amorphous and crystalline PLA extracts resulted in 228- and 319-fold increases, respectively, in IL-6 protein expression compared to untreated macrophages. Similarly, exposure of macrophages to lactic acid resulted in elevated IL-6 protein expression by 2.3-fold. Amorphous PLA extracts increased MCP-1, TNF-α, and IL-1β levels by 1.2-fold, 21-fold, and 567-fold, respectively. Likewise, crystalline PLA extracts increased MCP-1, TNF-α, and IL-1β levels by 4.7-fold, 27-fold, and 1,378-fold, respectively. Abnormally increased levels of IL-6, MCP-1, TNF-α and IL-1β were modulated by addition of 3PO, 2DG or aminooxyacetic acid. (FIGS. 21a-d, respectively) Levels of IFN-γ and IL-13 were unchanged by PLA extract (data not shown) but exposure to amorphous PLA extract decreased IL-4 protein levels by 3-fold relative to untreated macrophages. (FIG. 21e) With the exception of 3PO, IL-10 expression was either unchanged (crystalline PLA) or increased by 3.4-fold (amorphous PLA) upon addition of aminooxyacetic acid relative to macrophages exposed to PLA extract. (FIG. 21f).

Amorphous PLA was compounded with 2DG at 190° C. for 3 mins in a DSM 15 cc mini-extruder (DSM Xplore) and pelletizer (Leistritz Extrusion Technology). Our in-vitro studies indicate 1 mM 2DG to be an effective concentration. Accordingly, we estimated that 18 mg of 2DG in 10 g of amorphous PLA will approximate effective concentrations after accounting for potential thermal degradation of 2DG, converting mM to w/w values. We compounded comparable amounts (200 mg) of hydroxyapatite (HA; 2.5 μm particle sizes; Sigma-Aldrich) in 10 g of amorphous PLA under the same melt-blending thermal conditions. To exclude the effect of melt-blending as a confounder in our studies, amorphous PLA controls were processed under the same thermal conditions to make “reprocessed” amorphous PLA. Pellets from melt-blending were made into 1.75 mm diameter filaments using an extruder (Filabot EX2) at 170° C. with air set at 93. For surgical implantation, amorphous PLA filaments were cut into 1 mm lengths; four biomaterials were subcutaneously implanted on the dorsum (back) of each mouse, with two cranially (2.5 cm apart) and two caudally (2.5 cm apart).

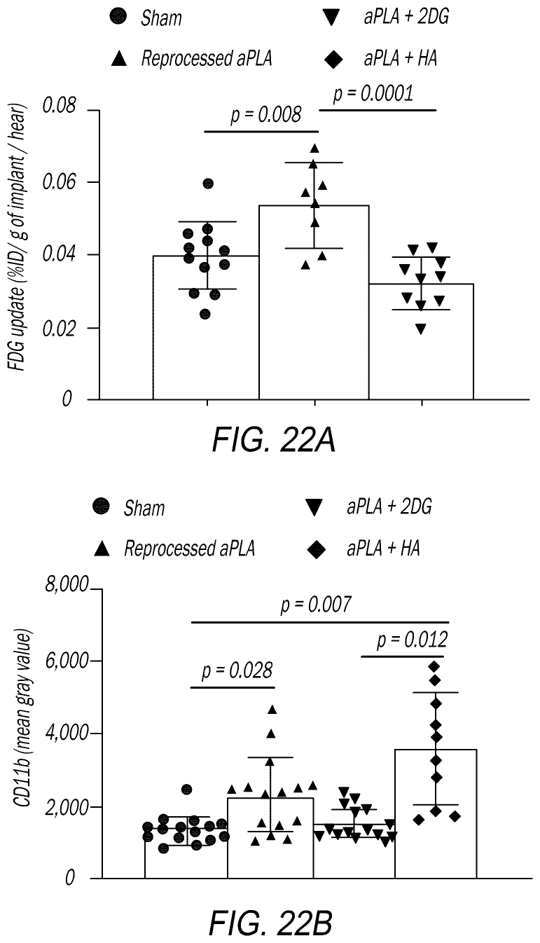

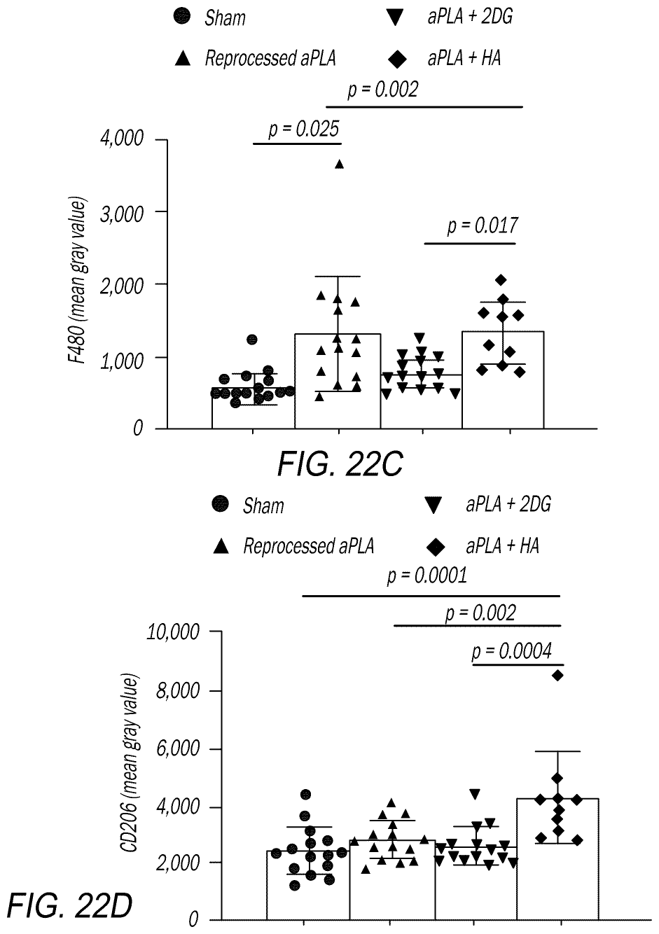

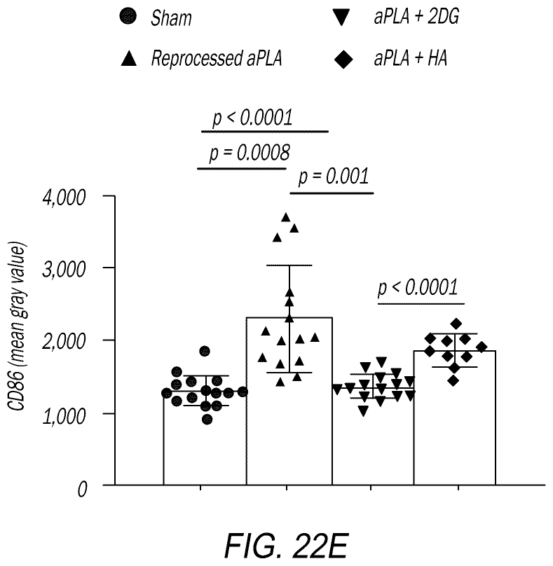

Two-month old female C57BL/6J mice (n=3 mice per group) with an average weight of 19 g were used according to procedures approved by the Institutional Animal Care and Use Committee at Michigan State University. Mice were anesthetized using isoflurane (2-3%). The back of each mouse was shaved and alternate iodine and alcohol swabs were used as skin disinfectants. Aseptic surgery consisted of incisions through the skin into the subcutis, where biomaterials were inserted into a pouch made with forceps. Afterwards, surgical glue (3M Vetbond) was used to appose the skin. Each mouse received intraperitoneal or subcutaneous pre- and post-operative meloxicam (5 mg/kg) injections as well as postoperative saline. Sham controls underwent the same procedure without biomaterial implantation. After 6 weeks, the dorsum of mice was shaved to visibly observe sites of surgical implantation. Thereafter, mice were intraperitoneally injected with 4.82 MBq F-18 fluorodeoxyglucose (Cardinal Health) in 200 μL. At 65 mins post-dose, mice were euthanized and blood drawn from their hearts. Circular biopsies (12 mm diameter) of full skin thickness, with visible implants in the center, were recovered. Similar sized biopsies were collected from mice in the sham group in the region where surgical incision was made. Biomaterial migration from subcutaneous sites only allowed for the recovery of most and not all implants. As such, for obtaining data on the gamma counter (FIG. 22a), there were 12 skin biopsies from 3 mice in the sham group, 8 skin biopsies from 3 mice (amorphous PLA group) and 10 skin biopsies from 3 mice (amorphous+2DG group). Skin biopsies, blood sample and heart organs were weighed, with only skin samples fixed in 4% paraformaldehyde (PFA). Activity in all samples was assessed via gamma counter (Wizard 2, Perkin Elmer) once decayed to a linear range. All injected doses and gamma counter measurements were decay corrected to the same timepoint to calculate the percent of injected dose taken up per gram of assessed tissue (% ID/g; FIG. 22a)

For tissue staining, one skin biopsy per mouse was passed through increasing concentration of 10%, 20% and 30% sucrose, daily. Using 99.9% methanol (Sigma-Aldrich) on dry ice, tissues were embedded in optimal cutting temperature (O.C.T.) compound (Tissue-Tek) by snap freezing. After equilibration at −20° C., multiple successive 8 μm sections were obtained using a microtome-cryostat. Sections were routinely stained using hematoxylin and eosin. Two different tissue sections were immunostained using conjugated antibodies as follows: 1) F4/80-FITC (1:100; BioLegend; 123107), CD11b-PE (1:100; BioLegend; 101207), CD206-BV421(1:200; BioLegend; 141717) and CD86-Alexa Fluor 647 (1:100; BioLegend; 105019) using ordinary mounting medium; 2) alpha-SMA-eFluor660 (1:150; ThermoFisher Scientific; 50-9760-82), TGF-β-PE (1:100; ThermoFisher Scientific; 12-9821-82) using DAPI mounting medium. Sections for TGF-β were permeabilized using 0.1% Triton X in 1×PBS (PBST) for 8 mins then washed off with 1×PBS generously. Afterwards, blocking buffer (0.5% bovine serum albumin in 1×PBS) was used to cover slides for 30 mins. Slides were then incubated in antibodies at 4° C. overnight. Subsequently, slides with tissue sections were washed in 1×PBS, and mounting medium applied.

Immunostained sections on slides were imaged using a Leica DMi8 Thunder microscope fitted with a DFC9000 GTC sCMOS camera and LAS-X software (Leica, version 3.7.4). Imaging settings at 20× magnification and 100% intensity were: 1) F4/80-FITC excitation using the 475 laser (filter 535/70; 500 ms); CD11b-PE excitation using the 555 laser (no filter; 500 ms); CD206-BV421 excitation using 395 laser (no filter; 150 ms); CD86-Alexa Fluor 647 excitation using the 635 laser (no filter; 500 ms). 2) alpha-SMA eFluor660 excitation using the 635 laser (no filter; 500 ms), TGF-beta-PE excitation using the 555 laser (no filter; 500 ms) and DAPI excitation using the 395 laser (535 filter; 500 ms). On the other hand, sections stained with hematoxylin and eosin were imaged at 40× using the Nikon Eclipse Ci microscope fitted with a CoolSNAP DYNO (Photometrics) and NIS elements BR 5.21.02 software (Nikon Instruments Inc.). Microscope images were prepared and analyzed using ImageJ (version 1.53k). For analyzing immunostained sections, 5 randomly selected rectangular areas of interest (1644.708 μm²), encompassing cells adjacent to implants, were obtained as mean gray values71 a tissue section. In the sham group, biopsies were taken from incision sites and areas without cells were also analyzed. Where derived from n=2 or n=3 mice, 10 or 15 data points, respectively are graphically represented to fully reveal inherent variance across samples; only the aPLA+HA group had sections derived from n=2 mice after one sample was damaged during cryo-sectioning and excluded from analyses. Representative images (16-bit; 0 to 65,535) were adjusted to enhance contrast for direct comparison using ImageJ as follows: CD86 (800-11,000), CD206 (2,000-5,000), F4/80 (500-4,000), CD11b (800-11,000), SMA (1,300-5,000), DAPI (6,000-31, 000), TGF (1,900-13,000).

### Example 2: Influence of PLA Stereochemistry on Immune Cellular Responses

This example demonstrates the immune responses to bioabsorbable polymer PLA of different stereochemistry. PLA containing >99% L-lactide (PLLA) and >99% D-lactide (PDLA) were obtained from NatureWorks, LLC as PLA L175 and PLA D120, respectively. To produce the 50/50 melt blend of PLLA and PDLA (also referred to as “stereocomplex PLA extracts”), premixtures of 50% PLLA and 50% PDLA were melt blended in a co-rotating twin-screw extruder type ZSE 27 HP-PH (Leistritz). The temperature profile range was 150-220° C. After quenching the filament in a cold-water bath, the product was pelletized and placed in a tray for drying for 24 hrs at 45° C. Prior to use, the PLA pellets were confirmed to be of similar curved surface area and sterilized by autoclaving at 121° C. for 20 minutes. Extracts were prepared by suspending 4 g of biomaterial pellets, having similar surface areas in 25 mL complete medium. After 12 days, at 250 rpm and 37° C., the medium containing PLA breakdown products (“extracts”) was decanted and used to treat cells for experiments. Control medium (without PLA pellets) underwent similar exposure. pH of extracts was assessed with an Orion Star A111 Benchtop PH meter at room temperature conditions (20° C.).

ATP levels in live cells were assessed by bioluminescence on the IVIS Spectrum in vivo imaging system (PerkinElmer) after addition of 150 μg/mL D-luciferin (PerkinElmer). For lysed cells, the standard ATP/ADP kits (Sigma-Aldrich) were used according to manufacturer's instructions.

MEF and BMDMs were prepared, maintained, and transfected as described in Example 1. Cell viability, OCR, ECAR, PER, cytokine levels, and chemokine levels were measured as described in Example 1.

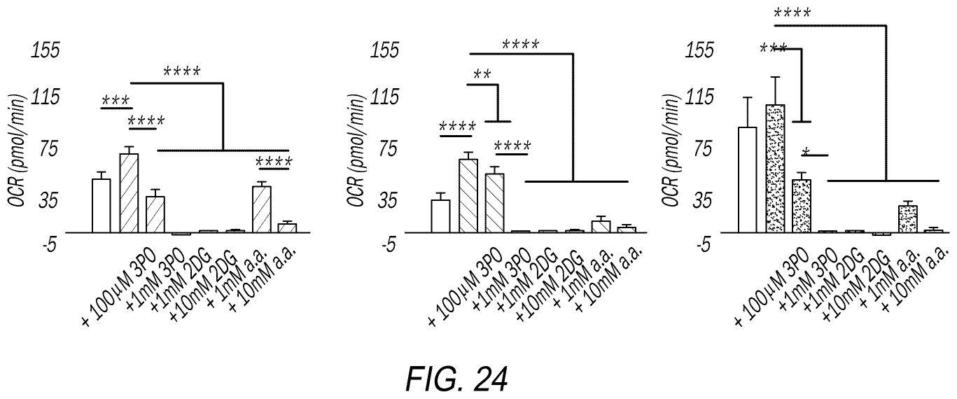

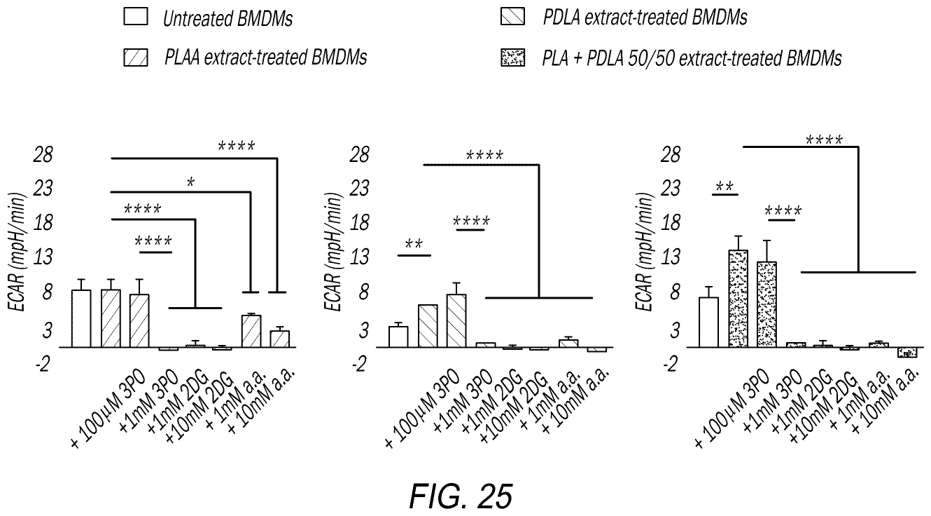

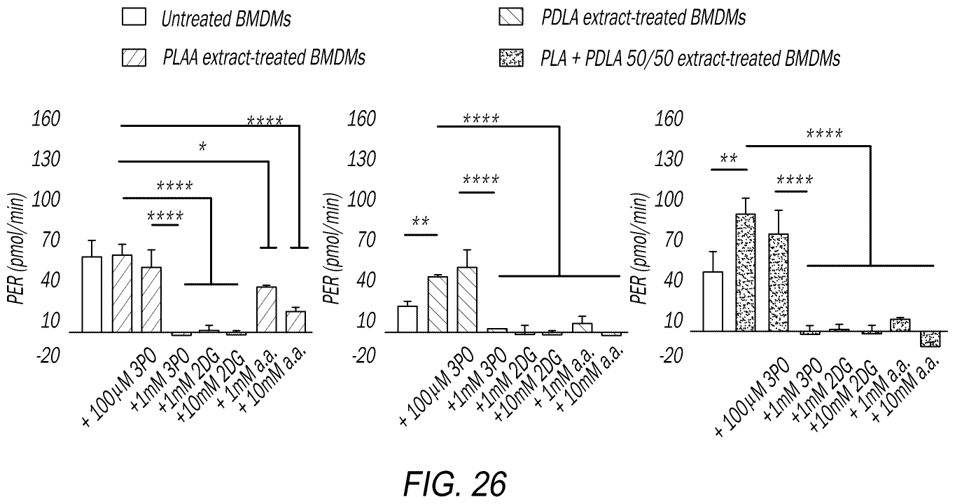

Initial studies showed that ATP levels in MEFs following exposure to PLLA, PDLA, and stereocomplex extracts were similar to that of untreated cells. (Data not shown.) However, BMDMs exposed to PLLA, PDLA, and stereocomplex PLA extracts expressed higher levels of ATP as compared to untreated cells on days 7 and 11. (Data not shown.) The functional metabolism of BMDMs exposed to PLLA, PDLA, and stereocomplex PLA extracts was measured. When compared to untreated macrophages, extracts of PLLA and PDLA increased OCR, but stereocomplex PLA extracts did not affect OCR. (FIG. 24) PDLA and stereocomplex PLA extracts increased ECAR and PER as compared to untreated macrophages, but PLLA did not induce a change. (FIGS. 25 and 26) Interestingly, the presence of metabolic inhibitors 3PO, 2DG, and a.a. induced a dose-dependent decrease in OCR, ECAR, and PER. (FIGS. 24-26)

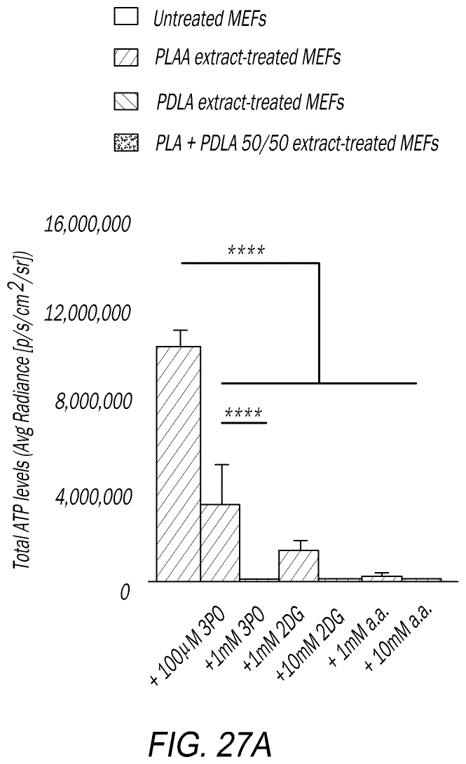

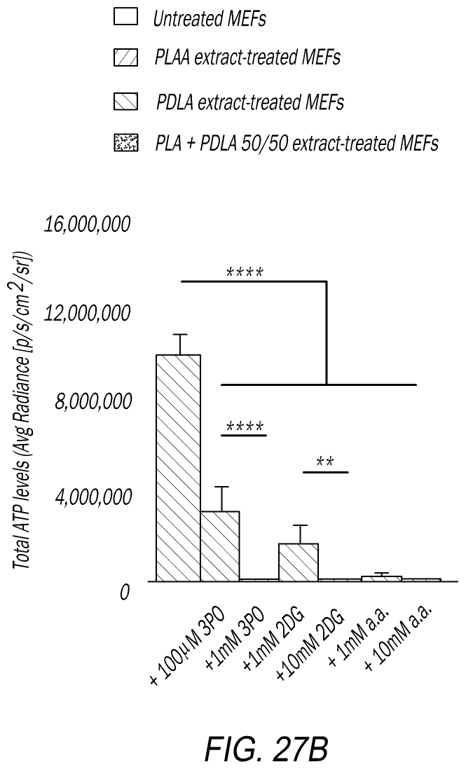

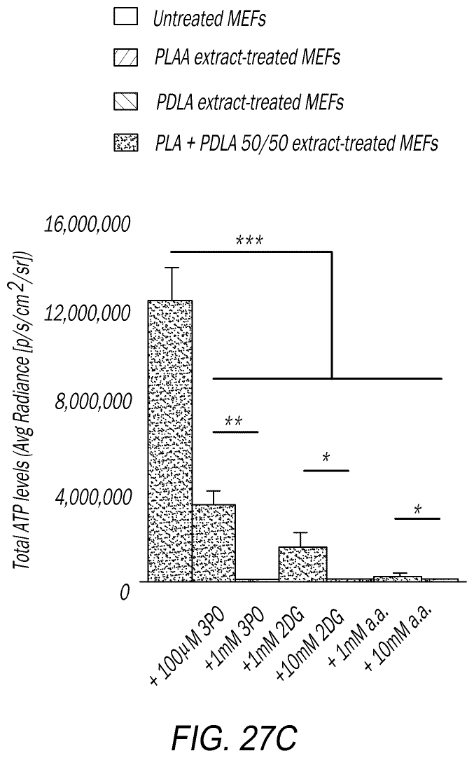

Total ATP levels were also measured in MEFs exposed to PLLA, PDLA, and stereocomplex PLA extracts in the presence of metabolic inhibitors. The ATP levels were decreased by the metabolic inhibitors 3PO, 2DG, and a.a. for MEFs exposed to each of PLLA, PDLA, and stereocomplex PLA extracts. (FIG. 27)

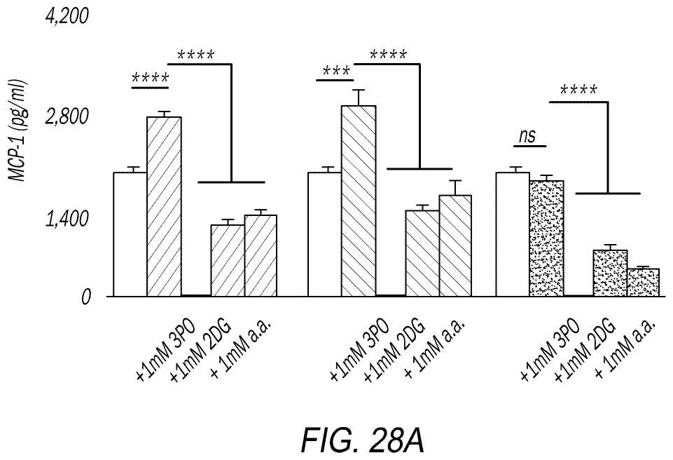

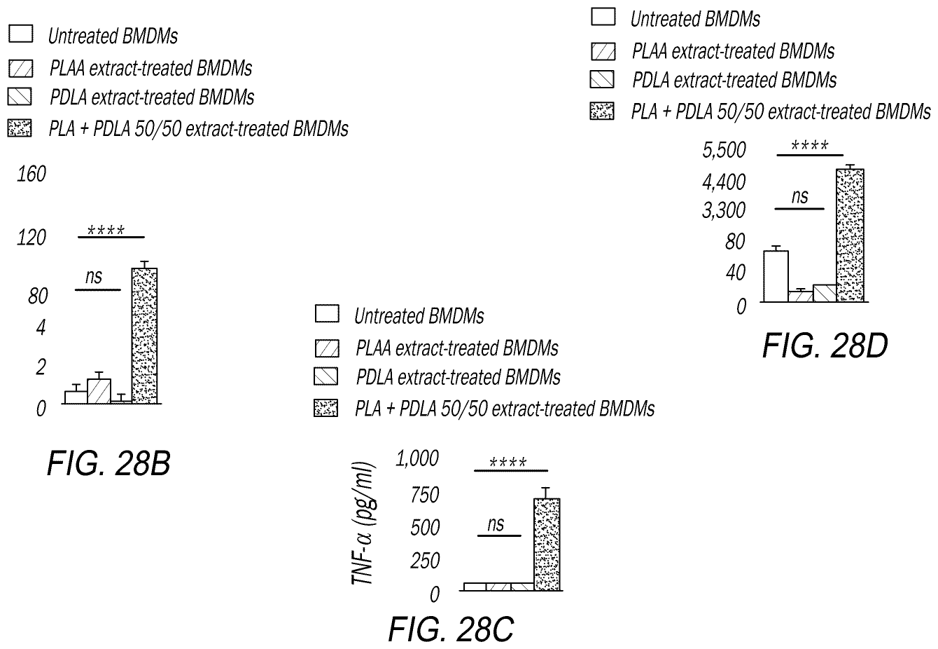

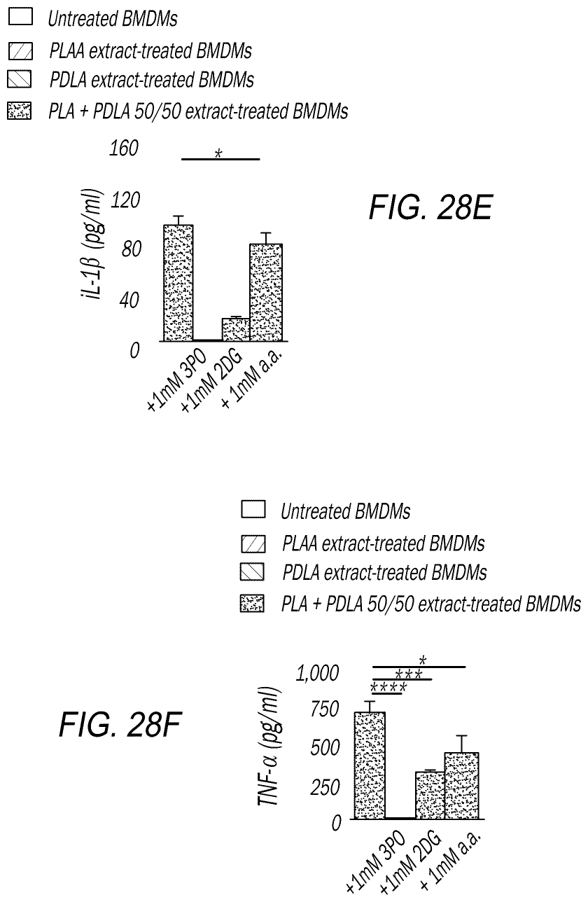

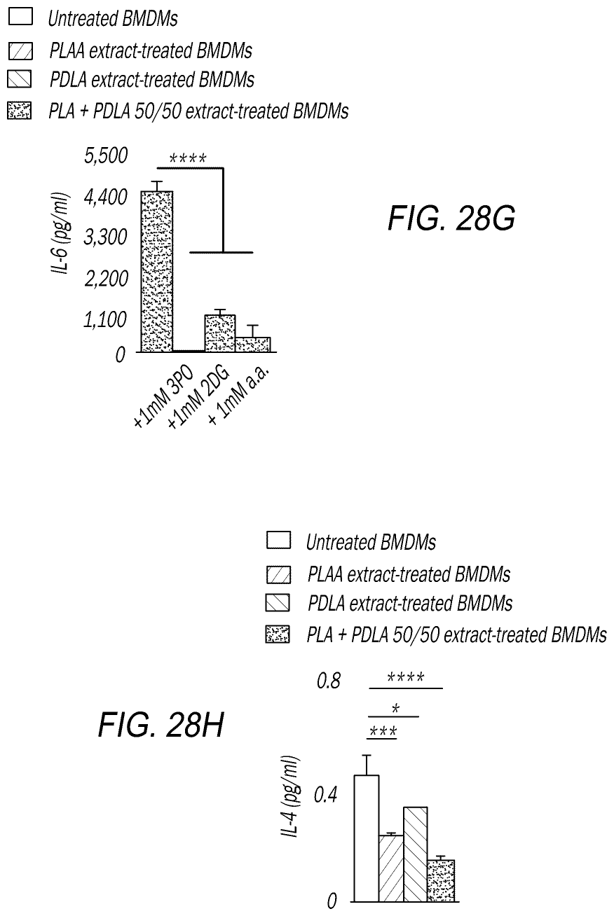

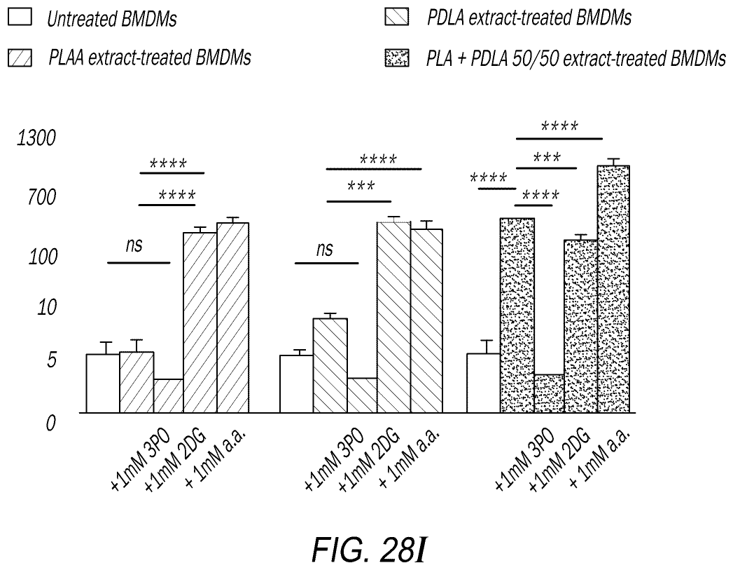

The altered bioenergetics caused by the PLA extracts has a direct effect on immune activation. Pro-inflammatory cytokines (e.g., MCP-1, IL-1β, TNF-α, IL-6, and IFN-γ) and anti-inflammatory cytokines (e.g., IL-4, IL-13, and IL-10) expression was measured. PLLA and PDLA extracts, but not stereocomplex PLA extracts, induced an increase in MCP-1 expression. Metabolic inhibitors successfully inhibited MCP-1 expression. (FIG. 28a) Stereocomplex PLA extracts, but not PLLA or PDLA extracts, significantly increased expression of pro-inflammatory cytokines IL-1β, TNF-α, and IL-6. (FIGS. 28b-d.) The enhanced cytokine expression in BMDMs treated with stereocomplex PLA extracts was blocked by metabolic inhibitors. (FIGS. 28e-g) There were no changes to IL-13 or IFN-γ expression. (Data not shown) Each of the PLA extracts inhibited IL-4 expression (FIG. 28h) but only the stereocomplex PLA extract had a statistically significant effect on IL-10 expression. (FIG. 28i) Addition of 2DH and a.a. increased IL-10 levels in macrophages exposed to the PLLA and PDLA extracts. Only a.a. further increased IL-10 levels in macrophages treated with stereocomplex PLA extracts.

The use of the terms “a” and “an” and “the” and “at least one” and similar referents in the context of describing the fusion peptide and related uses (especially in the context of the following claims) are to be construed to cover both the singular and the plural, unless otherwise indicated herein or clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Particular embodiments of the fusion peptide are described herein, including the best mode known to the inventors for carrying out the invention. Variations of those particular embodiments may become apparent to those of ordinary skill in the art upon reading the foregoing description. The inventors expect skilled artisans to employ such variations as appropriate, and the inventors intend for the fusion peptide to be practiced otherwise than as specifically described herein. Accordingly, the fusion peptide described herein includes all modifications and equivalents of the subject matter recited in the claims appended hereto as permitted by applicable law. Moreover, any combination of the above-described elements in all possible variations thereof is encompassed by the described fusion peptide unless otherwise indicated herein or otherwise clearly contradicted by context.

What is claimed is:

1. A composition comprising a polymer and a metabolic inhibitor,
wherein the polymer is selected from the group consisting of PLA, PLA-copolymer, PLGA, PGA, and combinations thereof, and
wherein the metabolic inhibitor is selected from the group consisting of a 6-phosphofructo-2-kinase/fructose-2,6-bisphosphatase 3 (PFKFB3) inhibitor, a glycolytic inhibitor, a biguanide, a γ-aminobutyrate aminotransferase (GABA-T) inhibitor, a mitochondrial electron transport chain (mETC) inhibitor, and combinations thereof,
wherein the composition comprises between about 1 μM and about 1 M of the metabolic inhibitor and
wherein the inhibitor comprises between about 0.01 wt % to about 30 wt % of the total weight of the composition.

2. (canceled)

3. The composition according to claim 1, wherein the composition comprises between about 0.5 mM and about 15 mM of the metabolic inhibitor.

4. (canceled)

5. The composition according to claim 1, wherein the metabolic inhibitor comprises between about 0.02 wt % to about 21 wt %, between about 0.01 wt % and about 17 wt %, between about 0.01 wt % and about 11 wt %, and between about 0.01 wt % and about 13 wt % of the total weight of the composition.

6. The composition according to claim 1, wherein the polymer is selected from the group consisting of PLA, PLGA, PGA, and combinations thereof.

7. The composition according to claim 6, wherein the PLA is selected from the group consisting of L-PLA, D-PLA, or a racemic mixture of PLA.

8. The composition according to claim 1, wherein the metabolic inhibitor is a PFKFB3 inhibitor.

9. The composition according to claim 8, wherein the PFKFB3 inhibitor is selected from the group consisting of 3-(3-pyridinyl)-1-(4-pyridinyl)-2-propen-1-one (3PO), (E)-1-(pyridin-4-yl)-3-(7-(trifluoromethyl)quinolin-2-yl)prop-2-en-1-one (ACT-PFK-158), (2S)-N-[[4-[3-cyano-1-[(3,5-dimethyl-4-isoxazolyl)methyl]-1H-indol-5-yl]oxy]phenyl]-2-pyrrolidinecarboxamide (AZ76), (2S)-N-[4-[[3-cyano-1-(2-methylpropyl)-1H-indol-5-yl]oxy]phenyl]-2-pyrrolidine carboxamide (AZ 26), and 1-(4-pyridinyl)-3-(2-quinolinyl)-2-propen-1-one (PFK15).

10. The composition according to claim 9, wherein the PFKFB3 inhibitor is 3PO and comprises between about 0.02 and 21 wt % based on the total weight of the composition.

11. The composition according to claim 1, wherein the metabolic inhibitor is a glycolytic inhibitor.

12. The composition according to claim 11, wherein the glycolytic inhibitor is selected from the group consisting of 2-deoxyglucose (2DG), 3-bromopyruvate, 3-fluoro-1,2-phenylene bis(3-hydroxybenzoate) (WZB 117), 4-[[[[4-(1,1-dimethylethyl)phenyl]sulfonyl]amino]methyl]-N-3-pyridinylbenzamide (STF 31), phloretin, quercetin, dichloroacetate, oxamic acid, and NHI1.

13. The composition according to claim 12, wherein the glycolytic inhibitor is 2DG and comprises between about 0.01 and about 17 wt % based on the total weight of the composition.

14. The composition according to claim 1, wherein the metabolic inhibitor is a biguanide.

15. The composition according to claim 14, wherein the biguanide is selected from the group consisting of metformin, buformin, and phenoformin.

16. The composition according to claim 15, wherein the biguanide is metformin and comprises between about 0.01 and about 11 wt % based on the total weight of the composition.

17. The composition according to claim 1, wherein the metabolic inhibitor is a GABA-T inhibitor.

18. The composition according to claim 14, wherein the GABA-T inhibitor is selected from the group consisting of aminooxyacetic acid, vigabatrin, gabaculine, phenelzine, phenylethylidinehydrazine (PEH), rosmarinic acid, valproic acid, ethanolamine-O-sulfate (EOS), and cycloserine.

19. The composition according to claim 15, wherein the GABA-T inhibitor is aminooxyacetic acid and comprises between about 0.01 and about 13 wt % based on the total weight of the composition.

20. The composition according to claim 1, wherein the metabolic inhibitor is an mETC inhibitor.

21. The composition according to claim 20, wherein the mETC inhibitor is selected from the group consisting of rotenone, rotenol, deguelin, dehydrodegulein, tephrosin, sumatrol, oligomycin, azithromycin, clarithromycin, erythromycin, and trifluoromethoxy carbonylcyanide phenylhydrazone.

22. The composition according to claim 21, wherein the mETC inhibitor is oligomycin and comprises between about 0.0001 and about 13 wt % based on the total weight of the composition.

23. The composition according to claim 1, wherein the metabolic inhibitor is incorporated into the polymer.

24. The composition according to claim 1, wherein the metabolic inhibitor is coated onto the polymer.

25. (canceled)

26. (canceled)

27. The composition according to claim 1, further comprising an additional therapeutic agent selected from the group consisting of chemotherapies for cancer, antibiotics, small molecules, antibodies, antigens, calcium phosphate, hydroxyapatite, and bioglass.

28. (canceled)

29. A method for modulating an immune response comprising providing to a subject in need thereof a composition comprising a polymer and a metabolic inhibitor,
wherein the polymer is selected from the group consisting of PLA, PLA-copolymer, PLGA, PGA, and combinations thereof, and
wherein the metabolic inhibitor is selected from the group consisting of a 6-phosphofructo-2-kinase/fructose-2,6-bisphosphatase 3 (PFKFB3) inhibitor, a glycolytic inhibitor, a biguanide, a γ-aminobutyrate aminotransferase (GABA-T) inhibitor, a mitochondrial electron transport chain (mETC) inhibitor, and combinations thereof,
wherein the composition comprises between about 1 μM and about 1 M of the metabolic inhibitor and
wherein the inhibitor comprises between about 0.01 wt % to about 30 wt % of the total weight of the composition.

30. (canceled)

31. (canceled)

32. (canceled)

33. The method according to claim 29, wherein the composition is a synthetic tissue or a depot.

34. The method according to claim 33, wherein the synthetic tissue is selected from the group consisting of bone, cartilage, tendon, skin, blood, kidney, and liver.

35. (canceled)

36. The method according to claim 29, wherein the subject is selected from the group consisting of a surgery subject, an orthopedic subject, an ophthalmologic subject, a subject suffering from a chronic disease, and injury.

37. (canceled)

38. (canceled)

39. A synthetic tissue comprising a polymer and a metabolic inhibitor,
wherein the polymer is selected from the group consisting of PLA, PLA-copolymer, PLGA, PGA, and combinations thereof, and
wherein the metabolic inhibitor is selected from the group consisting of a 6-phosphofructo-2-kinase/fructose-2,6-bisphosphatase 3 (PFKFB3) inhibitor, a glycolytic inhibitor, a biguanide, a γ-aminobutyrate aminotransferase (GABA-T) inhibitor, a mitochondrial electron transport chain (mETC) inhibitor, and combinations thereof,
wherein the composition comprises between about 1 μM and about 1 M of the metabolic inhibitor and
wherein the inhibitor comprises between about 0.01 wt % to about 30 wt % of the total weight of the composition.

40. The synthetic tissue according to claim 39, wherein the synthetic tissue is selected from the group consisting of bone, cartilage, tendon, skin, blood, kidney, and liver.

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)
